(19) United States
(12) Patent Application Publication
(10) Pub. No.: US 2021/0251605 A1
(43) Pub. Date: Aug. 19, 2021

(54) ULTRASOUND IMAGING PROBE

(71) Applicant: UCL BUSINESS LTD, London (GB)

(72) Inventors: Adrien DESJARDINS, London (GB); Richard COLCHESTER, London (GB); Malcolm FINLAY, London (GB); Charles Alexander MOSSE, London (GB); Sacha NOIMARK, London (GB); Ioannis PAPAKONSTANTINOU, London (GB)

(73) Assignee: UCL BUSINESS LTD, London (GB)

(21) Appl. No.: 17/252,637

(22) PCT Filed: Jun. 14, 2019
(86) PCT No.: PCT/GB2019/051658

§ 371 Date: Dec. 15, 2020

(30) Foreign Application Priority Data
Jun. 15, 2018 (GB) 1809841.8

(51) Int. Cl. A61B 8/00; A61B 8/12; G01S 15/89

(52) U.S. Cl. CPC A61B 8/4461; A61B 8/12; G01S 7/52079; A61B 8/4483; G01S 15/8965

(57) ABSTRACT

An ultrasound probe comprises an optical light guide comprising a multi-mode optical waveguide for transmitting excitation light and a single-mode optical waveguide for transmitting interrogation light. The probe further comprises an ultrasound transmitter located at a distal end of the probe, the ultrasound transmitter comprising an optically absorbing material for absorbing the excitation light from the multi-mode optical waveguide to generate an ultrasound beam via the photoacoustic effect. The probe further comprises an ultrasound receiver including an optical cavity external to the single-mode optical waveguide. The interrogation light from the single-mode optical waveguide is provided to the ultrasound receiver. The optical cavity has a reflectivity that is modulated by impinging ultrasound waves. The interrogation light is reflected from the optical cavity to a proximal end of the single-mode optical waveguide where it can be received for generating a signal. At least a portion of the ultrasound probe is configured to rotate so that the ultrasound beam is transmitted in a rotating direction.

### FIELD

The present application relates to an ultrasound imaging probe and to a medical instrument and ultrasound system incorporating such a probe.

### BACKGROUND

Ultrasound imaging systems have been used to view structures of medical relevance within the human body such as tissues and implants and are important for various diagnostic and other purposes. Ultrasound images derive contrast based on the mechanical properties of a medium (e.g. tissue) through which ultrasound propagates, including also the properties of any boundary or interface between two different media. In some ultrasound imaging systems, an ultrasound probe that transmits and/or receives ultrasound is applied externally to the body, while in other systems, the ultrasound probe is applied internally to the body.

Miniaturised ultrasound devices have found use in coronary procedures, where imaging can help to characterise plaque morphology. Conclusions can be drawn from the images provided as to whether/how to proceed with any intervention, including stent sizing and guiding placement. Many existing ultrasound imaging systems include transducers, consisting of one or more elements, connected to transmit and receive electronics. When miniaturising these electronic elements for use in intraluminal imaging, there are several challenges, including the dicing and connectorising of small elements, and achieving the sensitivity required whilst also having a high bandwidth for high resolution imaging. In addition, electrical ultrasound probes are susceptible to electromagnetic interference and so generally cannot be used at the same time as other procedures such as radio-frequency ablation and magnetic resonance imaging (MRI).

An alternative to generating and receiving ultrasound electronically is to use light. These optical systems have the advantage of being agnostic to the device size, as the generated ultrasound bandwidth is dependent on the optical pulse and not the lateral dimensions of the ultrasound element. Additionally, the lack of electronics provides an immunity to electromagnetic interference, and so may allow such devices to be used in a wider range of circumstances.

To generate ultrasound with light, the photoacoustic effect is typically used, whereby pulsed or modulated light is incident on an optically absorbing medium. Absorption of the light within the medium leads to rapid heating, causing a temperature rise and thus a corresponding pressure rise, which propagates as ultrasound. To receive ultrasound with light, interferometric methods are typically used, which rely on the impinging (incident) ultrasound causing a variation in the path length of light propagating in the receiver. By monitoring the change in path length, the incident ultrasound can be measured. One known implementation of an ultrasound receiver is a Fabry-Pérot hydrophone, in which a small optical cavity is formed on the end of an optical fibre (such as disclosed in (WO2001001090A1). Ultrasound impinging on the cavity changes the cavity thickness, which can be measured by a change in the reflection coefficient of the cavity. Another known implementation of an ultrasound receiver is a polymer optical ring resonator (such as disclosed in US20080095490A1).

A further advantage of optical ultrasound imaging is that the absorbing medium used to generate ultrasound can be configured to also transmit light (of a different wavelength), and this transmitted light may then be used for other imaging and sensing modalities. For example, Noimark et al. demonstrate the use of composite materials to acquire co-registered ultrasound and photoacoustic images, providing both structural and molecular contrast (see—Noimark, S., Colchester, R. J., Poduval, R. K., Maneas, E., Alles, E. J., Zhao, T., Zhang, E. Z., Ashworth, M., Tsolaki, E., Chester, A. H., Latif, N., Bertazzo, S., David, A. L., Ourselin, S., Beard, P. C., Parkin, I. P., Papakonstantinou, I. and Desjardins, A. E. (2018), Ultrasound Generation: Polydimethylsiloxane Composites for Optical Ultrasound Generation and Multimodality Imaging (Adv. Funct. Mater. September 2018). Adv. Funct. Mater., 28: n/a, 1870055. doi:10.1002/adfm.201870055).

One known approach for miniaturising optical sensors is to utilise fibre optic technologies, such as those used in the telecommunications industries. These often have the advantage of low manufacturing costs and established industrial processes. In addition, they may be available with a sufficient flexibility and small size (diameter) so as to be suitable for introduction into a catheter or other medical (e.g. intracoronary) device. As described in WO2016113543A1 and U.S. Pat. No. 6,519,376B2, small fibre optics can be used for the efficient transmission of light to and from the distal end of such a device, and imaging can be achieved with the provision of an ultrasound receiver at the distal end. As described in U.S. Pat. No. 7,245,789B2, a fibre Bragg grating may be used to direct light onto a photoacoustic ultrasound transmitter located on the side of an optical fibre.

There is a continuing interest in enhancing the imaging quality and capabilities of such optical ultrasound imaging systems.

### SUMMARY

The invention is defined in the appended claims.

An ultrasound probe comprises an optical light guide comprising a multi-mode optical waveguide for transmitting excitation light and a single-mode optical waveguide for transmitting interrogation light. The probe further comprises an ultrasound transmitter located at a distal end of the probe, the ultrasound transmitter comprising an optically absorbing material for absorbing the excitation light from the multi-mode optical waveguide to generate an ultrasound beam via the photoacoustic effect. The probe further comprises an ultrasound receiver including an optical cavity external to the single-mode optical waveguide. The interrogation light from the single-mode optical waveguide is provided to the ultrasound receiver. The optical cavity has a reflectivity that is modulated by impinging ultrasound waves. The interrogation light is reflected from the optical cavity to a proximal end of the single-mode optical waveguide where it can be received for generating a signal. At least a portion of the ultrasound probe is configured to rotate so that the ultrasound beam is transmitted in a rotating direction.

This ultrasound probe utilises a single optical light guide for both transmission and reception. Note that the fidelity of single mode supports higher quality reception; conversely, for transmission, single mode is limited in optical power for the excitation light, so multimode is used.

In another implementation, a sensor is provided for integration into an elongated medical device for performing ultrasound imaging from within the medical instrument. A single optical light guide is used in the sensor for both multimode excitation light and single mode reception light. Ultrasound is generated using the multimode light which is extracted from the light guide (which may be an optical fibre). Ultrasound generation occurs within an optically absorbing material. Ultrasound reception occurs using the single mode light within an optical cavity external to the optical light guide, such as a Fabry-Pérot cavity.

In another implementation, a sensor is provided for integration into an elongated medical device for performing ultrasound imaging from within the medical instrument. Ultrasound generation is performed at a distal end of a multimode excitation light guide using photoacoustic excitation. The direction of ultrasound transmitted into the surrounding medium is rotated with respect to the device longitudinal axis. Ultrasound reception is performed optically with an element positioned externally to a single mode interrogation light guide which is stationary with respect to the rotating ultrasound transmission element.

In another implementation, a device is provided for ultrasound imaging within a cavity or luminal body. The device comprises of one or more light guides configured to transmit pulsed or modulated light to a distal coating to generate ultrasound and to rotate about the longitudinal axis of the device, thus insonating a plane around the transmitter. An ultrasound receiver is included to perform imaging. The distal end of the device may be housed in an ultrasonically transparent material (such as TPX—a.k.a. polymethylpentene). Ultrasound is generated/directed such that it propagates predominantly perpendicular to the longitudinal axis of the light guide. Ultrasound generation occurs in an absorbing medium that may be attached to the light guide, or fabricated on a mechanically separate element. Ultrasound may be received efficiently perpendicular to the light guide longitudinal axis. Ultrasound transmitted by the device is reflected by physical structures and the reflected ultrasound may be received. Thus, an ultrasound A-line (single depth scan) can be acquired perpendicular to the light guide longitudinal axis, which gives information about the distance between the device and boundaries beside it. By rotating the transmitting element several A-lines can be acquired at different angles, and these can be reconstructed into a two-dimensional ultrasound image. By rotating the transmitted through 360° and acquiring A-lines at regular intervals, the entire surrounding environment can be imaged. To create a three-dimensional image, the entire device can be moved along the longitudinal axis of the light guide, whilst also rotating the transmitter.

In some implementations, an ultrasound catheter is provided for generating and receiving intraluminal ultrasound images. The ultrasound catheter comprises one or more optical fibres with distal ends sensorised such that ultrasound can be transmitted and received by the device. The catheter has an elongated body for insertion into luminal structures, such as a blood vessel. The one or more optical fibres are configured to allow rotational ultrasound imaging without rotation of the outer catheter housing. At the distal end, the catheter is connected to a sled which controls rotation of the one or more optical fibres and can pull back the device for 3D helical imaging.

Note that although various implementations having various features are described herein (both above and below), the skilled person will appreciate that features from different implementations can be combined as appropriate to create new or different implementations, or to enhance or extend the various implementations.

An optical ultrasound imaging device such as described herein may be used to image within cavities or luminal bodies, and may also be extended for use in a wide range of other physiological and biological situations, including detection, measurement and therapy, such as laser ablation. The imaging may be performed in a clinical context, such as for the guidance and assessment of stent placement in coronary arteries, and/or to image luminal structures, such as blood vessels within a living body, for example, for use in characterising atherosclerotic plaque in blood vessels.

A fibre optic ultrasound imaging device (probe) as described herein is able to provide both high depth penetration and high resolution in a rotational fashion, creating a two or three-dimensional image of a lumen or cavity. The ultrasound probe may be integrated into an invasive medical device, such as a stent delivery device.

As noted above, in some implementations, a transmitter optical light guide and a receiver optical light guide may be implemented (fabricated) as a single optical guide, such as a dual-clad optical fibre. Excitation light for generation of ultrasound may be redirected out of the light guide, without affecting light guided for the interrogation of the receiver. The transmission light may be redirected, for example, by using a refractive index gradient, removal of the second cladding in a dual clad fibre, use of a fibre Bragg grating, and/or fabrication of a wavelength selective mirror within the fibre (amongst others).

The light for generating the ultrasound may be redirected a short distance prior to the distal end of the ultrasound probe. In this case, the ultrasound transmitting element is generally close to the ultrasound receiving element (within a couple of millimetres). This configuration helps to support smaller lateral device dimensions (<1 mm), thereby allowing the device to be used in more tightly confined locations.

In some implementations, the absorbing medium used to generate ultrasound may be wavelength selective. In other words, light of a specific wavelength (or wavelength range) may be absorbed, and light of an alternative wavelength (or wavelength range) may be transmitted. This medium or material may consist, for example, of metallic nanoparticles, organic dyes or quantum dots, amongst others, to provide the wavelength selectivity. The light which is transmitted through the optically absorbing material into the surrounding medium (tissue) may be absorbed in the tissue, where it will generate ultrasound via the photoacoustic effect. This ultrasound can be received using the same ultrasound receiver as the reflected ultrasound resulting from the excitation light that is absorbed within the optically absorbing material, and can be used to provide molecular contrast. This approach may be extended to include spectroscopic imaging, which enables the calculation of physiological parameters, such as blood oxygenation. This approach therefore allows for multimodality imaging, in which photoacoustic and ultrasound imaging are carried out simultaneously. Alternatively, the transmitted light could be used for other imaging and sensing methods, or for therapy, such as optical ablation.

The excitation light for generating ultrasound may be generated by a short pulsed optical source, which will generate a wide-bandwidth ultrasound pulse. Alternatively, the generation light may be modulated, allowing precise control over the generated ultrasound frequency and bandwidth. This allows a choice between high resolution, low depth imaging and low resolution, high depth imaging, whilst using low cost laser components to generate the ultrasound. These different ultrasound formats may allow for the differentiation between objects in the image depending on the relative reflection of high and low frequency ultrasound. In addition to depth tuning, this method may also be used to encode the ultrasound with a known pattern or sequence, thereby enabling the use of various noise reduction methods, or removing the limitation of ultrasound transit time on the acquisition speed.

In some implementations, the ultrasound probe is configured to provide both a view ahead of the probe and a circumferential two-dimensional image. In such a device, a wavelength selective mirror may applied to a 45° distal end surface of the light guide used for transmitting ultrasound. One wavelength of light is directed perpendicularly to the fibre axis by the mirror, impinging upon an optically absorbing medium and generating ultrasound for the 360° two-dimensional imaging. A second wavelength of light is transmitted through the mirror and impinges on another optically absorbing medium, thereby generating ultrasound ahead of the device along the longitudinal axis of the light guide. This latter ultrasound may be used for guiding the device placement and/or distance sensing to help avoid collision with delicate surfaces.

As with a conventional electrical ultrasound system, if a single pulse of ultrasound waves is transmitted into tissue, then a series of echos are typically obtained, at delay times T1, T2, etc., compared with the transmission of the original pulse (assumed to be at time T=0). The echo at time T1 is a reflection from a structure within the tissue, for which the ultrasound travel time from the transmitter (the optical absorbing coating) to the tissue and then back to the receiver totals T1. If we assume that the speed of the ultrasound waves in the body is V, and that the optical absorbing coating and the optical element are close enough together so as to be considered as spatially coincident at the distal end, then the depth (distance) D of a reflecting structure from the distal end 103 with a delay T1 is given by V/2T.

To create an image that is able to differentiate between ultrasound reflections from different directions, the ultrasound is either transmitted or received from a small angular aperture (given by solid angle Δϕ that is allowed to rotate 360 degrees around the instrument circumference. In some implementations, an omnidirectional ultrasound receiver, such as a Fabry-Perot fibre optic sensor, is used in conjunction with a highly directional ultrasound transmitter. In such implementations, only the ultrasound transmitting component may be rotated. If a series of pulse-echo measurements are acquired and concatenated in a display, with the vertical axis representing depth into tissue and the horizontal axis representing time, an M-mode image is obtained. Such an M-mode image may be suitable for certain clinical applications but unsuitable for others. Furthermore, an image may be produced by rotating the direction of the ultrasound projection (beam) with respect to the longitudinal (Z) axis to create a two-dimensional image of the surrounding tissue, similar to that of a conventional B-mode ultrasound image, in which the depth into the tissue and orientation (rotational angle) of reflections with respect to the optical absorbing coating can be established.

Rotation of the ultrasound beam provides scans of concentric circles each having a radius defined by multiplication of the ultrasound beam transmission depth, from the probe, and the sinusoid of ϕ, where ϕ represents an offset angle between the direction of the ultrasound transmission and the rotational axis. If the offset angle is tightly defined and close to 90 degrees, relative to the longitudinal (and rotational) axis Z, then the radius corresponds to the transmission depth.

It is noted that existing devices may use a single optical fibre including one or more Bragg gratings for lateral transmission and reception of ultrasound. However, such devices tend to have the following limitations:
Fibre Bragg gratings generally have poor ultrasound reception sensitivity due to the stiffness of the material they are built into, i.e. the optical fibre, which makes them unsuitable for imaging applications which require high sensitivity to receive low pressure reflections from tissue.
Interrogation for ultrasound reception may involve the use of a single mode core of an optical fibre. However, the use of the same single mode core (typically with a small diameter of 5 to 10 microns) for excitation light is unsuitable for the pulse energies for the generation of ultrasound beams that are substantially collimated over distances of many mm, with intensities in the MPa range.

In contrast, the approach described herein utilises a cavity external to the optical fibre, such as a Fabry-Pérot hydrophone, to achieve higher sensitivity and signal strength. Furthermore, to combine ultrasound transmission with ultrasound reception via such an external Fabry-Pérot cavity, a multimode light guide is used to accommodate excitation light pulses with sufficient pulse energies to achieve ultrasound pressures and corresponding signal strengths suitable for high quality imaging applications. This multimode light guide can be provided by having separate optical fibres for transmission and reception, or by using a multicore or dual clad optical fibre, which has both multimode and single mode optical channels within a single fibre.

In some implementations, an omnidirectional ultrasound receiver can be used. This allows the receiver to remain stationary whilst the transmitter is rotated. One advantage of using a rotating transmitter and a stationary receiver is that cross-talk (direct ultrasound transmission from the transmitter to receiver) will be dependent on the transmitter angle, and so the angle of the transmitter can be recovered for image reconstruction based at least in part on the measured level of cross-talk. Furthermore, when separate fibres are used for transmission and reception, allowing the receiving optical fibre to remain stationary avoids problems associated with having to rotate two separate fibres (such as the risk of the fibres becoming wound together, the difficulty of coupling light into both rotating fibres in an efficient manner, and how to synchronise the rotation so that the transmitter and receiver face the same direction).

Although some implementations described herein utilise a Fabry-Pérot hydrophone for the ultrasound receiver, other components can be used for receiving ultrasound reflected from tissue depending upon the circumstances of any given implementation. For example, in some implementations, the ultrasound receiver may be a microring optical resonator.

### BRIEF DESCRIPTION OF THE DRAWINGS

Various implementations of the invention will now be described in detail by way of example only with reference to the following drawings:

FIG. 1 is a schematic diagram of an ultrasound system in accordance with some implementations of the invention.

FIGS. 2A and 2B are schematic sectional diagrams of examples of ultrasound transmitters such as may be used (inter alia) in the ultrasound system of FIG. 1.

FIGS. 3A and 3B are schematic sectional diagrams of further examples of ultrasound transmitters such as may be used (inter alia) in the ultrasound system of FIG. 1.

FIGS. 4A and 4B are schematic diagrams of the distal end of an example of a medical instrument including an ultrasound transmitter and an ultrasound receiver such as may be used (inter alia) in the ultrasound system of FIG. 1, with FIG. 4A providing a side section and FIG. 4B providing an end view.

FIG. 5 is a schematic diagram of an example drive device for rotating an optical light guide such as may be used (inter alia) in conjunction with the ultrasound system of FIG. 1.

FIGS. 6A and 6B are schematic sectional diagrams of further examples of ultrasound transmitters such as may be used (inter alia) in the ultrasound system of FIG. 1, in which only a transmitting element (or a portion thereof) is rotated, rather than the length of an optical light guide.

FIGS. 7A and FIG. 7B are schematic diagrams showing a side view and an end view, respectively, of an example of a single optical fibre that provides an ultrasound transmitter and receiver pair such as may be used (inter alia) in conjunction with the ultrasound system of FIG. 1.

FIGS. 8A, FIG. 8B, and FIG. 8C are schematic diagrams showing a side view (A, C) and an end view (B), of further examples of a single optical fibre which provides an ultrasound transmitter and receiver pair such as may be used (inter alia) in conjunction with the ultrasound system of FIG. 1.

FIG. 9 is a schematic diagram of an example of a dual-modality ultrasound and photoacoustic transmitter such as may be used (inter alia) in conjunction with the ultrasound system of FIG. 1.

FIG. 10 is a schematic diagram of another example of an ultrasound transmitter such as may be used (inter alia) in conjunction with the ultrasound system of FIG. 1.

FIG. 11 is a schematic diagram of the ultrasound transmitter shown in FIG. 3A being used in conjunction with two (differently modulated) beams of excitation light.

FIG. 12 is a schematic diagram of another example of an ultrasound transmitter such as may be used (inter alia) in conjunction with the ultrasound system of FIG. 1.

FIG. 13 is a schematic diagram of a console such as may be used (inter alia) in conjunction with the ultrasound system of FIG. 1.

### DETAILED DESCRIPTION

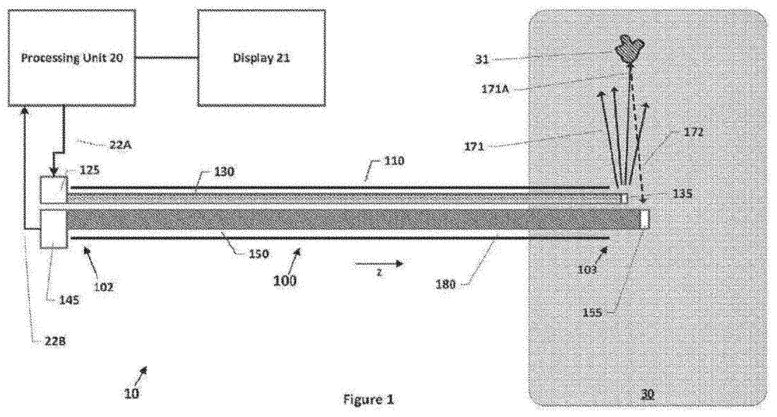

FIG. 1 is a schematic diagram of an ultrasound imaging system 10 in accordance with certain implementations of the invention. The imaging system includes a processing unit 20 with an associated display monitor 21, and a medical instrument 100 comprising an ultrasound imaging probe, which may be implemented as (or incorporated into) a stent delivery device, a needle stylet or cannula, an endoscopic probe, or any other appropriate device.

The instrument 100 has a proximal end 102 and a distal end 103 and includes (i.e. incorporates, or has integrated into it) a first optical light guide 130 and a second optical light guide 150 (also referred to herein as optical guides), each of which extends from the proximal end to the distal end. The processing unit 20 has appropriate optical and/or electrical connections to the proximal end 102 of the instrument 100. The distal end 103 of the instrument is shown located in tissue 30 and includes a facility for generating and transmitting ultrasound into the tissue, and also an ultrasound receiver for receiving ultrasound from tissue. The arrow Z, which extends in the direction from the proximal end 102 to the distal end 103 of the medical instrument, can be considered as representing the primary or longitudinal axis of the medical instrument 100.

A first electrical link 22A from the processing unit 20 is joined to the instrument 100 by a coupling device 125, e.g. an electro-optical coupler, which in turn connects to the first optical light guide 130. This electro-optical coupler 125 includes a light source for providing excitation light that has a time-varying pattern in accordance with an electronic control signal provided from the processing unit 20 via electrical link 22A. For example, the control signal may be configured to provide a continuous light source or may be configured to create a specific temporal pattern of light, such as a sequence of pulses. In other implementations, link 22A may be used to provide an optical input signal to the instrument 100 (via some suitable optical coupling).

The first optical light guide 130 conveys the excitation (illumination) light from the proximal end to the distal end of the instrument (in other words it acts as a transmission optical light guide). In some cases, the processing unit 20 may be able to generate excitation light of two or more different wavelengths, either at the same time or in (potentially rapid) succession.

In some implementations, the first optical light guide may be provided as an optical fibre (a transmitter fibre), for example, as a multimode fibre that extends along the instrument 100. The second optical light guide 150, as described in more detail below, may also be implemented as an optical fibre (either the same optical fibre as for the first optical light guide, or a different optical fibre). The use of optical fibres for the first and/or second optical light guides 130, 150 supports the fabrication of a medical device 100 (such as a length-agnostic flexible catheter) having a small diameter, thereby supporting intra-vascular use and other similar applications.

The first and second optical light guides 130, 150 are housed within, and protected by, a polymer housing tube 110 or sheath (or similar) which forms an external surface of the probe 100, perpendicular to the longitudinal axis. The light guides 130, 150 are housed within such polymer tubing to help avoid damage to/from (and interference with) the surrounding tissue 30. In some examples, the polymer housing 110 may extend beyond the distal end 103 of the first and/or second optical light guides 130, 150 (not shown). In such an implementation, the polymer housing 110 may be substantially transparent to ultrasound (at least at the distal end of the medical instrument 100). For example the polymer housing 110 may be polymethylpentene (TPXTM), which is transparent to ultrasound. Furthermore, each light guide 130, 150 may be individually housed within a housing tube (not shown) or sheath, which may be constructed in a similar manner to housing tube 110.

In some examples, there is space surrounding the optical fibres 130, 150 (within the polymer housing tube 110) to allow a fluid column, e.g. saline (not shown in FIG. 1), to be established along the length of the instrument 100. This space can be provided in different ways: for example, by using a dedicated tubular structure, or by using a region surrounding the stylet within the cannula. The presence of the fluid allows the pressure at the distal end 103 of the instrument 100 to be measured at the proximal end 102. For example, the proximal end 102 may be linked to a manometer in order to measure and record the pressure at the distal end 103 of the instrument 100. Additionally, the fluid may act to provide cooling to components at the distal end 103 and/or to drive rotation of components at the distal end 103 (as described in more detail below).

The ultrasound imaging system 10 uses excitation light travelling along the first optical light guide 130 to generate ultrasound at the distal tip of the instrument 100. For example, excitation light from the coupling device 125 is transmitted along the optical light guide 130 from the proximal end 102 to the distal end 103 to generate ultrasound using the photoacoustic effect. Thus as shown in FIG. 1, the excitation light travels down the first optical guide 130 to the distal end 103 and is incident on (and absorbed by) an optically absorbing coating 135. The resulting thermal deposition into the optically absorbing material of the coating 135 converts the light into ultrasound 171 that propagates from the instrument 100 into the tissue 30 as shown by arrows 171. The light provided along the first optical light guide 130 is therefore regarded as excitation light, since it causes the coating 135 to generate (and emit) the ultrasound waves 171, i.e. the coating 135 acts as an ultrasound transmitter. Note that the ultrasound may be emitted (transmitted) in a direction which is substantially perpendicular to the longitudinal axis Z of the instrument 100 (as shown in FIG. 1). In addition, the excitation light travelling down the first optical light guide 130 may be pulsed or modulated in amplitude, which in turn produces a corresponding pulsing/modulation of the ultrasound generated by the optically absorbing coating 135.

The optically absorbing coating 135 may be formed from a material in an elastomeric host. For example, the optically absorbing material 135 may comprise an elastomer such as polydimethylsiloxane with integrated carbon nanotubes. The optically absorbing coating 135 may further comprise gold nanostructures integrated into a polymer (Xiaotian Zou, Nan Wu, Ye Tian, and Xingwei Wang, “Broadband miniature fiber optic ultrasound generator,” Opt. Express 22, 18119-18127 (2014)), carbon black (T. Buma, M. Spisar and M. O'Donnell, “Thermoelastic expansion vs. piezoelectricity for high-frequency, 2-D arrays,” in IEEE Transactions on Ultrasonics, Ferroelectrics, and Frequency Control, vol. 50, no. 8, pp. 1065-1068, August 2003), or graphite (E. Biagi, F. Margheri and D. Menichelli, “Efficient laser-ultrasound generation by using heavily absorbing films as targets,” in IEEE Transactions on Ultrasonics, Ferroelectrics, and Frequency Control, vol. 48, no. 6, pp. 1669-1680, November 2001). The skilled person will be aware of other suitable materials for forming the coating 135.

In some implementations, the optically absorbing coating 135 may be applied directly onto the end of the optical light guide 130. In other implementations, as described below, the optically absorbing coating 135 may be provided to a region (or the whole) of the circumference of the optical guide 130, or may be provided to a region (or the whole) of the circumference of the medical instrument 100 at its distal end. (The circumferential direction typically lies within a plane perpendicular to the longitudinal axis Z of the medical instrument 100).

The processing unit 20 may be used to control the temporal distribution (time variation) of the ultrasound 171 emitted from the ultrasound transmitter at the distal end 103 of instrument 100 (i.e. from the optically absorbing coating 135 for the implementation shown in FIG. 1). In particular, the processing unit 20 may control the temporal distribution of ultrasound waves from the ultrasound transmitter by sending appropriate control signals to the electro-optical coupler 125 to produce a desired temporal pattern, for example, a sequence of pulses, for the excitation light travelling along the first optical light guide 130. The temporal pattern of the emitted ultrasound 171 then generally matches or follows the temporal pattern of the excitation light.

As described in more detail below, the distal end 103 of the first optical fibre 130 is configured to propagate ultrasound into the medium 30 in a direction which is at an angle to (i.e. offset from) the longitudinal axis Z of the medical device 100 (and likewise to the longitudinal axis of the first optical fibre 130). In particular, the ultrasound transmitter of the probe 100 generally supports the directional transmission of ultrasound into the tissue 30. In some cases, the offset angle (φ) may be of the order of 90 degrees, so that ultrasound 171 is propagated in a direction which is substantially perpendicular to the longitudinal axis Z. Such a propagation direction may be regarded as providing a side view from the medical instrument 100, as opposed to a forward view (where the ultrasound would travel further in the direction of longitudinal axis Z).

In other cases, the offset angle (φ), may be less than 90 degrees, for example, in various implementations, φ may be greater than 15 degrees, greater than 30 degrees, greater than 45 degrees, greater than 60 degrees, greater than 75 degrees, or in the range 15-30 degrees, 30-45 degrees, 45-60 degrees or 60-75 degrees. Some implementations of instrument 100 may support the use of multiple different offset angles. Note that different offset angles produce different viewing directions, which may be helpful to image particular anatomical structures (for example, because of limitations in the available positioning of the distal end 103 of the probe 100, and/or because of potential obstructions that may prevent imaging at other offset angles).

It will be appreciated that in practice, the ultrasound beam generated by an apparatus such as probe 100 may have some degree of divergence. Nevertheless, the direction of the beam (such as for determining offset angle (φ) can be determined according to any suitable measure, such as the geometric centre of the beam, an intensity-weighted centre of the beam, the direction of peak intensity (luminosity), etc.

In some examples, the excitation light may be redirected at the distal end 103 by using an angled mirror (reflecting surface), such that the light leaves the optical light guide 130 through a side surface. Outside the light guide, the excitation light may then impinge upon an optical absorbing medium, which may be shaped to further influence the ultrasound directivity, such as the spread and/or offset angle of the beam. The optically absorbing medium (material) may be directly coated onto the first light guide 130 (analogous to coating 135 as shown in FIG. 1, but located on the side, rather than an end, of the first light guide). Other possibilities include that the optically absorbing medium is located on a separate housing or another medium (not shown in FIG. 1).

As illustrated in FIG. 1, ultrasound waves 171 transmitted from the optically absorbing coating 135 may be (partially) reflected by one or more anatomical structures 31 within the tissue 30, such as a tumour or the inner surface of a heart atrium. At least some of the reflected waves 172 may return towards the instrument 100 and impinge on an optical element 155 which is located at the distal end 103 of the second optical light guide 150. This optical element 155 acts as a transducer or ultrasound receiver (e.g. hydrophone) to convert the ultrasound waves 172 that are incident on the optical element 155 into a corresponding optical signal which is propagated by the second optical light guide from the distal end 103 to the proximal end 102 of the instrument 100. The hydrophone may, for example, be a concave Fabry-Pérot fibre optic hydrophone, which receives interrogation light over the second optical light guide, the reflection of this interrogation light back along the second optical light guide being dependent on the spacing of the Fabry-Pérot hydrophone (which is turn is dependent on the ultrasound signal incident on the receiver). In other implementations, the receiving element may operate to receive ultrasound electronically, such as by using piezoelectric or capacitive micro-machined transducers.

The receiving element (such as hydrophone 155) and the transmitting element (such as coating 135) are generally in close colocation with each other (any separation being <1 mm). In other implementations, the transmitting element and the receiving element may have a translational offset (along the Z axis), e.g. a separation>1 mm with respect to one another at the distal end 103 of the instrument; one reason for having such an offset is to avoid (or reduce) the receiver 155 occluding the transmitter 135, and conversely to avoid (or reduce) the transmitter 135 occluding the receiver 155. Such a configuration can also help to reduce the direct signal at the receiver, i.e. the ultrasound that directly propagates from the transmitter 135 to the receiver 155 without undergoing any reflection in tissue 30.

In some implementations, some form of shielding, e.g. a hypotube, may be placed between the receiver 155 and the transmitter 135 to reduce the direct signal, i.e. to help isolate the optical element 155 from ultrasound transmissions that would otherwise propagate directly to it from the optically absorbing coating 135, or at least, such that the direct ultrasound transmissions are significantly attenuated. In some implementations, this isolation or attenuation may be accomplished by positioning the optical element 155 in a metal hypotube, so that the optical element is recessed from the distal end of the hypotube. In some implementations, alternative (or additional) isolation or attenuation may be obtained by positioning the optically absorbing coating in a second metal hypotube, so that the coating 135 is recessed from the distal end of the second hypotube.

The receiver 155 generates an output optical signal in response to the magnitude and phase of the incident ultrasound waves, together with the variation in time of the phase and magnitude (but the receiver 155 does not, in itself, provide any direct imaging or sensing of the spatial distribution of the ultrasound waves). The output optical signal conveyed along the second optical light guide (optical guide) 150 from the distal end 103 to the proximal end 102 therefore incorporates temporal variations that derive from the temporal variations of the ultrasound waves 172 incident onto the optical element 155 (hence the second optical light guide 150 may be referred to as a receiving fibre or similar). This output optical signal, including the modulations thereof, is then converted into an electrical signal by electro-optical coupler 145 for return to the processing unit 20 for analysis and display, etc.

The skilled person is aware of various possible implementations for the electro-optical coupler 145. In some implementations, the electro-optical coupler 145 includes a fibre-coupled wavelength-tunable light source that provides interrogation light to the second optical light guide 150 via a circulator; the light received from second optical light guide 150 is provided to a photodetector. It will be appreciated that electro-optical coupler 145 may be a somewhat different type of device from electro-optical coupler 125 (as described in more detail below), since generally the former may be used to perform an electrical->optical conversion, whereas the latter may be used to perform the reverse conversion.

As described above, the ultrasound 171 generated by transmitter 135 propagates away from the device and may be reflected by objects 301 in the surrounding environment (tissue 30). In some implementations, the first optical light guide 130 may be rotated around the longitudinal axis Z; assuming a non-zero offset angle (such as a 90 degree offset angle shown in FIG. 1), this then allows the receiver 155 to receive reflections from objects which have different azimuthal (circumferential) directions with respect to the rotational axis of the first optical light guide. In effect, such rotation therefore enables the medical instrument 100 to acquire an ultrasound scan around the plane perpendicular to the rotational axis of the first optical light guide (for an offset angle of approximately 90 degrees). More generally, the scan region defines the surface of a cone having a half angle of φ.

In some implementations, the receiver 155 is largely omnidirectional (isotropic), whereby its response has little or no sensitivity to the direction of the incoming ultrasound wave 172. Consequently, the receiver is able to receive sound reflected from different angles without being moved or rotated, at least for the range of directions of interest for receiving reflected ultrasound waves 172. This range depends primarily on the relative locations of the transmitter 135 and receiver 155 for the instrument 100, the offset angle discussed above of the emitted ultrasound beam, the shape of the ultrasound beam (e.g. the amount of any divergence), and the depth of propagation of the ultrasound beam within the tissue 30. If the direction of maximum sensitivity within this range is considered to represent 100%, then one way to characterise the level of omni-directionality is by stating that at least A % of the range has a sensitivity greater than B %, where A may equal (for example), 60%, 80%, 90%, 95% or 100%, and B may equal (for example) 50%, 60%, 70%, 80% or 90%. (The skilled person will be aware of other ways of characterising this omni-directionality).

An ultrasound image may be acquired using probe 100 by rotating the transmitter 360 degrees about (around) the longitudinal axis Z and receiving reflections from various angles in the (omnidirectional) receiver 155. For example, in an implementation such as shown in FIG. 1, in which the ultrasound 171 is transmitted in a direction substantially perpendicular to the rotational axis of the first optical light guide 130 (and hence substantially perpendicular to the longitudinal axis Z), we can define positions in a plane by polar coordinates (r, θ) centered on the distal end of the instrument 100. For any given reflected signal 172 received by the receiving element 155, the angle θ is known according to the rotational angle of the first optical light guide when the outgoing ultrasound signal 171 was emitted, while the distance r can be determined from the time delay between the emission of the outgoing ultrasound 171 from the transmitter 135 and receipt of the reflected ultrasound signal 172 at the receiver 155 (assuming a known ultrasound propagation speed in tissue 30 and that any displacement between the transmitter 135 and receiver 155 is small or can be otherwise allowed for).

In some implementations, the rotational angle θ may be determined (for example) using an encoder at the proximal end of the first optical light guide 130. Another possibility is that the rotational angle θ is calculated from measuring the strength of the direct ultrasound signal (as transmitted directly from the transmitter to the receiver without reflection), since this will generally vary according to the direction of the transmitted ultrasound 171 compared to the direction from the transmitter 135 to the receiver 155.

The above approach acquires a two-dimensional ultrasound image corresponding to the plane of (r, θ). In some implementations, the first optical light guide 130 (or more generally, the direction of propagation for the outgoing ultrasound signals 171) may be rotated around the longitudinal axis Z at 60 Hz to give a corresponding frame rate of 60 Hz. However, in other implementations, the rotation may be faster or slower. In some implementations, the distal end 103 of the instrument 100 may be moved (shifted) along the longitudinal axis Z (e.g. using a continuous or stepped motion). This motion then effectively provides a set of planar slices at successive positions along the Z axis to build up a three-dimensional (volumetric) image.

In some implementations, the transmitter 135 may be rotated about the Z axis by rotating the proximal end of the first optical light guide 130 which supports the transmitter 135. For example, the first optical light guide 130 may be rotated at the proximal end 102 using a motor; this can be facilitated by providing a rotary junction to avoid twisting of the first optical light guide 130. In addition (or alternatively) torque transmission along the first optical light guide 130 may be assisted by using a torque (torsion) coil surrounding the first optical light guide.

In some implementations, the transmitting element located at the distal end 103 of the instrument 100 may be separate from (the majority of) the first optical light guide 130 so as to allow the transmitting element at the distal end 103 to be rotated (about the Z axis) in effect independently (i.e. decoupled from) the first optical light guide 130. For example, the transmitter 135 may be located in an optical head which is rotationally decoupled from the remainder of the first optical light guide 130. Rotation of the optical head may be achieved, for example, using a micro-electronic motor, or a hydraulic motor, which uses the flow of a fluid over a turbine to rotate the transmitter. In another implementation, energy from the transmitted ultrasound may also be used to rotate the transmitter. Another possibility is that light of the same or a different wavelength (compared to that of the excitation light) may be transmitted through the first optical light guide 130 to activate a micro-electronic motor. It will be appreciated that the skilled person may utilise other techniques to power and control rotation of the emitted ultrasound beam.

Accordingly, a combination of (i) temporal variation (e.g. pulsing and/or modulation) of the transmitted ultrasound, and (ii) rotational movement (scanning) of the direction of propagation of the transmitted ultrasound can be used to facilitate synthesis of a 2-D ultrasound image of the tissue 30. Furthermore, by also utilising (iii) translational motion of the distal end of the instrument (or at least of the transmitting and receiving elements), a 3-D ultrasound image of the tissue 30 may be acquired.

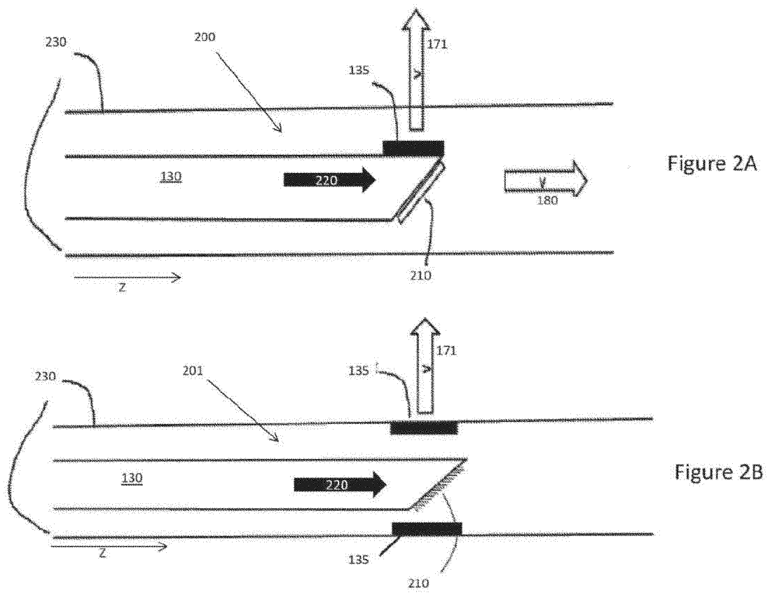

FIG. 2A shows an example ultrasound transmitter 200 such as for use in the instrument 100 illustrated in FIG. 1 (note that for clarity, the ultrasound receiver is not shown in this diagram). The transmitter 200 is contained within a polymer housing tube 230. There may be a fluid or saline solution 180 flowing between the ultrasound transmitter 200 and the polymer tube housing 230. In some examples, the optical fibre 130 may have a single, consistent diameter along its entire length, while in other examples (not shown) the optical fibre may have a smaller diameter at the proximal end to facilitate manoeuvrability. This may be implemented by connecting the smaller diameter fibre to the larger fibre (with the ultrasound transmitter 200 at the distal end) via splicing, gluing (with epoxy or other), or any other suitable method.

The ultrasound transmitter 200 is configured to generate and emit an ultrasound beam 171 into a surrounding medium having a direction away (offset) from the longitudinal axis Z of the medical instrument 100. In the example shown in FIG. 2A, the emitted beam is (i) substantially parallel (collimated), and (ii) in a direction substantially perpendicular to the longitudinal (z) axis. Having a parallel beam helps to provide good image resolution, since the ultrasound beam 171 illuminates a well-defined and limited region of the tissue 30. In some cases, the beam may be converging (have a divergence angle less than zero). Having a beam perpendicular to the longitudinal direction helps to improve the signal-to-noise ratio of the ultrasound image, since the ultrasound beam 171 has to travel through less tissue to reach a given depth, and there is also more direct correlation between depth and signal travel time.

In some implementations, the beam may not be (i) substantially parallel and/or (ii) substantially perpendicular to the longitudinal axis. Note that if the beam is not substantially parallel, the direction of the beam can be specified by any suitable technique as discussed above, such as the geometric centre of the beam, or a weighted average based on summing the luminosity strength in each direction. For the reasons given above, the beam direction may be preferably offset from the longitudinal axis Z by at least 45 degrees, at least 60 degrees, at least 75 degrees, at least 80 degrees, at least 85 degrees, or approximately 90 degrees (as shown in FIG. 2A). Likewise, for the reasons given above, the beam is preferably contained within a divergence angle of less than 45 degrees, less than 30 degrees, less than 20 degrees, less than 15 degrees, less than 10 degrees, less than 5 degrees, or approximately parallel (within 2 degrees, 1 degree or less). Note that when determining beam divergence, the above divergence angle may be taken as applying to most rather than all of the beam, such as 75%, 90%, etc of the total beam luminosity; for example, 80% of the beam power falls within a divergence angle of less than 10 degrees.

In some implementations, the beam 171 may be converging (have a divergence angle less 0 degrees, i.e. a negative divergence). This can lead to a more concentrated and focussed beam, which can help to improve both image resolution, by having a smaller beam size, and also signal-to-noise ratio, by having a more concentrated beam (i.e. more ultrasound power per unit area).

In accordance with FIG. 1, the transmitter 200 shown in FIG. 2A comprises an optical light guide 130 (such as an optical fibre) and an absorbing coating or surface 135 for generating ultrasound. The optical fibre 130 serves as the excitation light guide and has an end surface angled at 45° to the longitudinal axis (Z), which is coated with a mirror 210 (optical reflector) at the distal end 103. The optical fibre 130 may be cleaved or polished (or otherwise manufactured) to create the angled end surface to which the optical mirror 210 is attached. The mirror 210 itself may be formed of a metallic film such as gold, a dielectric mirror, or a refractive index interface that provides total internal reflection, while the mirror coating (if provided) may be applied using any suitable technique, such as evaporation, sputtering and so on. The optically absorbing coating 135 is applied to the side of the fibre 130 (approximately opposite mirror 210) and may, for example, be a composite of polydimethylsiloxane, used for its high thermal expansion coefficient, and an absorbing material, such as carbon nanotubes. The coating 135 may be applied by dip coating or any other suitable method.

Excitation light 220 travelling along the optical fibre 130 impinges at the distal end upon the mirror 210, where the excitation light 220 is reflected and redirected to the edge of the fibre 130. The redirected light exits the optical fibre 130 and enters the optically absorbing coating 135, where the redirected light is absorbed. This absorption leads to the generation of an ultrasound wave 171, which propagates away from the transmitter. In some implementations, the optically absorbing coating 135 may have a shaped profile to focus the transmitted ultrasound 171, for example, to produce a parallel or converging beam as discussed above.

Thus in FIG. 2A, light travels from the proximal end of the optical fibre 130 to the distal end in a longitudinal direction, and is then reflected by mirror 210 to travel in a radial direction towards the absorbing surface 135 on the outside of the optical fibre 130, which then produces ultrasound beam 171 which is generated in a direction corresponding to that of the incident light (i.e. in a radial direction). Therefore, if it is desired to have the ultrasound beam 171 transmitted in a different direction into the tissue 30 (i.e. not exactly perpendicular to longitudinal axis, but rather, for example, at an offset angle of 80 degrees to the longitudinal axis), this can be arranged by having an end surface with a different angle—such as 40 degrees (rather than 45 degrees as shown in FIG. 2A).

FIG. 2B shows a further example of an ultrasound transmitter 201 for use in an instrument 100 such as described in FIG. 1 (note that for clarity, the ultrasound receiver is again not shown in this diagram). Many aspects of the implementation of FIG. 2B are the same as for FIG. 2A, and hence will not be described again for reasons of brevity. The main difference between the implementations of FIGS. 2A and 2B is that in the former, the optically absorbing coating 135 is formed on the outer surface of the optical light guide 130, whereas in the latter, the optically absorbing coating 135 is provided in conjunction with the external housing tube 230. For example, the optically absorbing coating 135 may be incorporated into the housing tube 230, or may be deposited onto the external or internal surface of the housing tube 230. As in FIG. 2A, the coating 135 may (for example) be a composite of polydimethylsiloxane, used for its high thermal expansion coefficient, and an absorbing material, such as carbon nanotubes, and the coating 135 may be applied by dip coating or any other suitable method.

As described above, the transmitter 201 produces a rotating ultrasound beam, such that the beam direction rotates about the longitudinal axis of the instrument 100. If the housing 230 co-rotates with the optical fibre 130, then the coating 135 can be provided at a single position on the housing, where the mirror 210 reflects the light that forms the ultrasound beam 171. On the other hand, if the optical fibre 130 rotates within the housing 230, such that the housing itself does not rotate, the coating may be provided in the form of a band wrapped around the circumference of the housing (in effect to define a short tube of coating 135 that is coaxial with housing 230). Accordingly, as the optical fibre 130 rotates within the housing, through various different azimuthal angles, the light exiting the optical fibre 130 after reflection by mirror 210 continues to impinge on the coating 135 (the band thereof) at the azimuthal position corresponding to the current rotational angle of the optical fibre in order to generate and emit ultrasound 171. FIG. 2B illustrates this latter configuration schematically, showing a section through the coating 135 located at both the top and bottom of the housing 230. Note that in some implementations, the coating 135 may be provided as a band even where the housing 230 and optical fibre 130 co-rotate with one another.

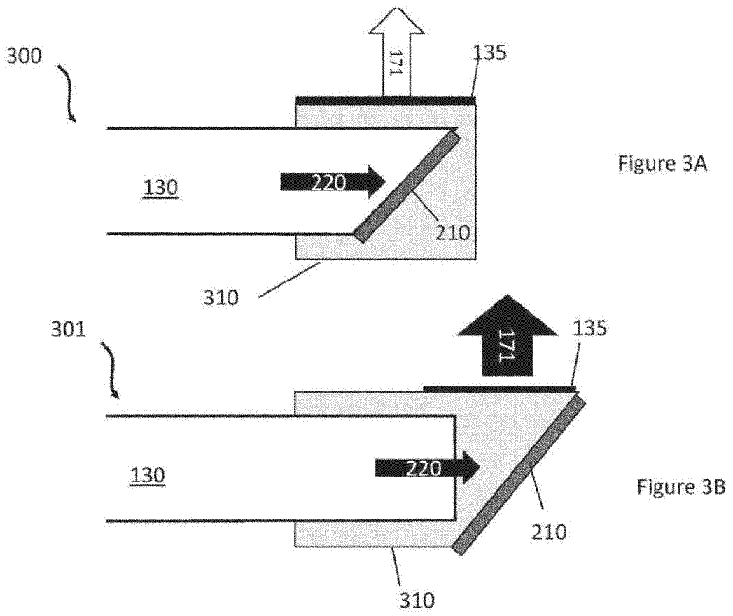

FIG. 3A shows a schematic sectional view of a further example of an ultrasound transmitter 300 such as for use in the instrument 100 illustrated in FIG. 1 (note that for clarity, the ultrasound receiver is again not shown in this diagram). Many aspects of the implementation of FIG. 3A are the same as for the ultrasound transmitters 200, 201 of FIGS. 2A and 2B, and hence will not be described again for reasons of brevity. The main difference between the implementation of FIG. 3A and the implementations of FIGS. 2A and 2B is that in FIG. 3A the transmitter 300 includes a housing 310 at the distal end of the fibre. The housing 310 may be made, for example, from an optically transparent epoxy, such as NOR81 (Norland). The housing 310 with the optical mirror 210 and absorbing surface 135 may be constructed separately from the optical fibre 130, with the latter being inserted into the housing 310 during a subsequent production step.

An optically absorbing coating 135 is provided on the surface of the housing 310, and excitation light is reflected (directed) as described above by the mirror 210 towards this coating 135 to produce ultrasound beam 171. As for the implementations described above, coating 135 may be a composite of polydimethylsiloxane and an absorbing material such as carbon nanotubes, and the coating 135 may be applied by dip coating or any other suitable method. In some examples, the optically absorbing coating 135 may have a shaped profile to focus the transmitted ultrasound 171.

Although FIG. 3A shows a mirror coating (metallic or otherwise) 210 to reflect the light towards the coating 135 as shown, in other examples the redirection may occur by total internal reflection (or other means) at the distal end of the optical fibre 130. Accordingly, the optical mirror 210 (optical reflector) may not be a separate component (as shown in FIG. 3A), but instead the surface of the housing 310 at the interface with the angled surface of optical fibre 130 may act inherently as a mirror due to the composition and relative refractive indices of the materials of the housing 310 and the optical fibre 130.

FIG. 3B shows a schematic sectional view of a further example of an ultrasound transmitter 301 such as for use in the instrument 100 illustrated in FIG. 1 (note that for clarity, the ultrasound receiver is again not shown in this diagram). Many aspects of the implementation of FIG. 3B are the same as for the ultrasound transmitter 300 of FIG. 3A, including the provision of housing 310, and hence will not be described again for reasons of brevity.

The main difference between the implementation of FIG. 3B and the implementation of FIG. 3A is that in FIG. 3B the distal surface of the housing 310 has a 45° angled surface or wedge, whereas the optical fibre 130 has a straight (perpendicular) distal end (which simplifies the production of optical fibre 130, since there is no need for further manufacturing to angle the surface of the distal end with respect to the main longitudinal axis of the optical fibre). The angled surface of the housing 310 is provided with (e.g. coated by) a mirror 210, which may be formed (for example) of a metallic film such as gold. In some cases, the mirror 210 may be implemented through the use of total internal reflection or any other suitable means (rather than by applying a metallic coating). The surface of the housing 310 to which excitation light is directed (reflected) by the mirror 210 is coated with an optically absorbing medium 135, such as described above.

The distal end of the optical fibre 130 of FIG. 3B may be manufactured before being inserted into or encompassed by housing 310, which may be formed from an epoxy (for example). Light from the optical fibre 130 is incident on the angled surface of the housing 310 and redirected by mirror 210 onto the coating 135 to generate an ultrasound beam 171. The use of an end cap 310, such as shown in FIG. 3A or 3B), may facilitate having a smaller optical fibre 130 while still providing a larger transmitter 310 for generating ultrasound.

The ultrasound transmitters 200, 201, 300, 301 in the example implementations shown in FIGS. 2A, 2B, 3A and 3B may be integrated into a medical device or instrument 100 together with an ultrasound receiver to support ultrasound imaging. Such an instrument 100 may be a very thin device, such as a guidewire. In some cases, such a transmitter 200, 201, 300, 301 may be integrated into an over-the-wire catheter, which may be placed into a vessel, organ or lumen.

The ultrasound beam produced by transmitters 200, 201, 300, 301 (as well as by those transmitters described below) may be focused with a concave ultrasound generating surface and/or with an ultrasound lens element (not shown in the Figures). Alternatively, the ultrasound beam may be expanded with a convex ultrasound generating surface and/or with an ultrasound lens element (not shown in the Figures).

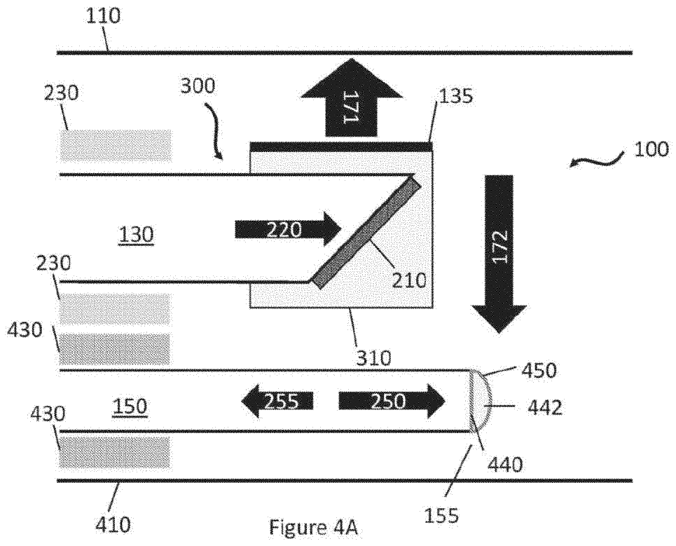

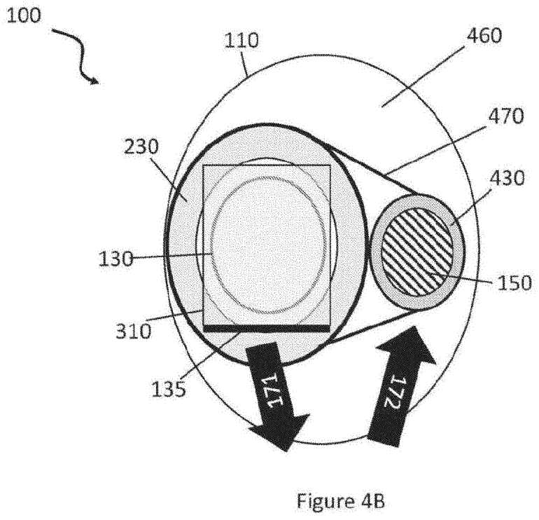

FIG. 4A and FIG. 4B are schematic diagrams showing a side view and an end view, respectively, of an example of an ultrasound transmitter and receiver pair at the distal end of a medical instrument 100 such as for use in an ultrasound imaging system 10 as described herein. The medical instrument 100 may be formed, for example, into a catheter to carry out intraluminal imaging, which can then be used (inter alia) to determine properties such as lumen diameter.

The instrument 100 of FIG. 4A includes an ultrasound transmitter 300, which generally corresponds to the ultrasound transmitter 300 shown in FIG. 3A and described above. However, in other implementations, transmitter 300 may be replaced with ultrasound transmitter 200, 201, or 301, such as shown in FIGS. 2A, 2B, or 3B respectively (or with any other suitable implementation, including those described further below).

The optical fibre 130 leading to transmitter 300 is housed in the lumen of a tube 230, with the distal end of a first optical fibre 130 (light guide), including the transmitter 300, exposed at the end of the tube 230. The tube 230 may be made of a polymer, such as fluorinated ethylene propylene, which has a low coefficient of friction to allow the optical fibre 130 (and hence the transmitter) to rotate easily within the instrument 100.

The device 100 also includes an ultrasound receiver, which is implemented as a Fabry-Pérot fibre optic hydrophone 155 located at the distal end of a second optical fibre 150 (light guide). The Fabry-Pérot fibre optic hydrophone 155 includes a planar mirror 440 at the end of the optical fibre 150. The Fabry-Pérot fibre optic hydrophone 155 further includes a transparent dome 442 formed on the mirror 440, and a curved mirror 450 formed on the outer (distal) surface of the dome 442. The mirrors 440, 450 may be formed from a metallic film such as gold. The dome may be formed using an epoxy and deposited by dip coating or any other suitable method.

The operation of Fabry-Pérot fibre optic hydrophone 155 is based on the combination of mirrors 440 and 450, which both reflect interrogation light received from the proximal end of optical fibre 150. The reflected interrogation light is subject to interference (constructive or destructive) according to the separation of the mirrors 440, 450 relative to the wavelength of the interrogation light. The separation of the mirrors 440, 450 is sensitive to ultrasound waves incident on the hydrophone 155, thereby allowing the incident (received) ultrasound to be measured using the interference pattern of the reflected interrogation light received at the proximal end of medical instrument 100.

The optical fibre 150 of the receiver is housed in tubing 430. The distal end of the optical fibre 150, including the hydrophone 155, is exposed at the end of the tube 430. The receiver 155 is generally sensitive to ultrasound received across the full range of azimuthal angles. Both the receiver 155 and transmitter 300, along with their respective tubings 430, 230, may be housed in an outer tubing 110. This housing may be made of an ultrasonically transparent material, such as TPX, to enhance the passage of ultrasound beam 171 out from the transmitter 300 into tissue 30, and the receipt of a return beam (reflected by tissue 30) at hydrophone 155.

Ultrasound imaging using instrument 100 may be performed by rotating the transmitter 300 within its tubing 230, thereby using the transmitted ultrasound beam 171 for azimuthal angular scans about the longitudinal axis of the instrument 100. The receiver 155 is kept stationary (not rotated), but generally provides 360 degree sensitivity, i.e. at substantially all azimuthal angles about the longitudinal axis of the instrument 100. Accordingly, receiver 155 is able to receive reflected ultrasound 172, such as may be reflected from anatomical object 31 (see FIG. 1).

FIG. 4B shows an end view of instrument 100 of FIG. 4A (as seen from the distal end). The instrument 100 comprises the outer polymer housing tube 110, in which two separate channels 230, 430 have been positioned for the transmitting fibre 130 and the receiving fibre 150 respectively. The channels 230, 430 have a circular profile with a diameter large enough to accommodate the respective optical fibres 130, 150. In some implementations, the instrument 100 may include a gap between the transmitter fibre 130 and the tube wall 230 and/or a gap between the receiver fibre 150 and its surrounding wall 430; this gap (or gaps) may be used for fluid flow, such as a saline solution 180 (for example, as discussed above in relation to FIG. 2A).

A heat shrink tubing 470 is shown in FIG. 4B and is used to hold the tubing 430 containing the receiver fibre 150 to the outside of the polymer tubing 230 housing the transmitter fibre 130 (or vice versa). As a result, the relative positions of the receiver fibre 150 and the transmitter fibre 130 are tightly controlled, to ensure consistent operation. Various other methods may be used for securing the relative positions of tubes 230, 430 (and hence fibres 130, 150) within the instrument 100, such as by forming tubes 230, 430 from a structure having a figure of “8” cross section that therefore provides two parallel lumens in a fixed configuration or by using multilumen tubing.

By way of example only, in some implementations, optical fibre 130 may have a diameter in the range 300-500 microns, while optical fibre 150 typically has a smaller diameter than optical fibre 130, for example, in the range 100-150 microns; the channel 230 has an external diameter in the range 500-900 microns, while the outer housing 110 has a diameter in the range 1-1.5 mm. It will be appreciated that these dimensions are illustrative only and not intended to be limiting; the specific sizing of any given implementation will depend on the overall design requirements and available technologies for use in the implementation.

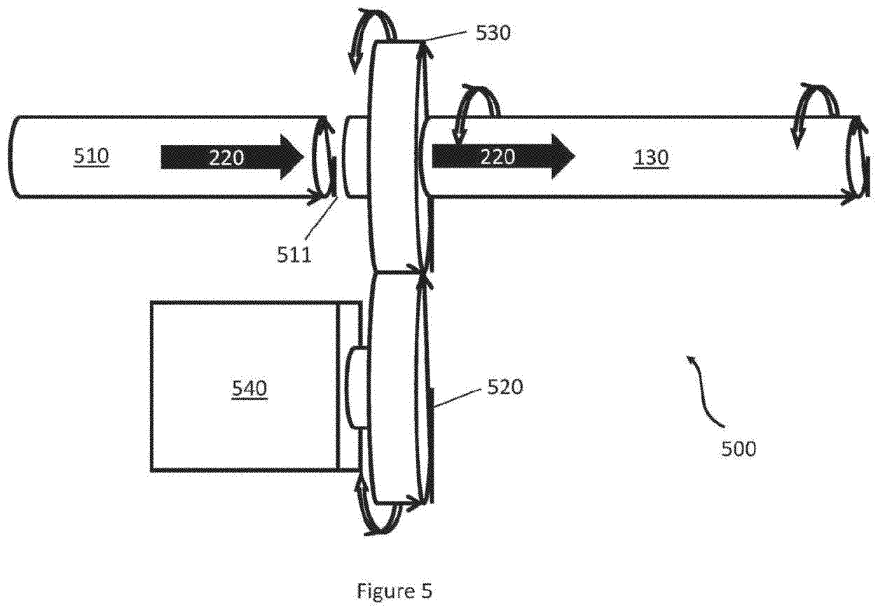

FIG. 5 is a schematic diagram of an example drive device 500 for rotating the first optical light guide 130 (such as an optical fibre), and hence also rotating the ultrasound transmitting element 200, 201, 300, 301 which is located at the distal end of the first optical light guide 130. Also shown in FIG. 5 is a proximal light guide 510, which is non-rotating, and used to provide excitation light 220 into the first optical light guide 130. In operation, excitation light 220 is transmitted through the proximal optical fibre 510, exiting the distal end of proximal optical fibre 510, and is then received into the (rotating) first optical light guide 130 for transmission to the ultrasound transmitting element 200, 201, 300, 301. To support this operation, the distal end of the proximal optical fibre 510 is located close to the proximal end of the first optical light guide 130, but typically there is a slight separation 511 to facilitate the rotation of the first optical light guide 130 relative to the proximal optical fibre 510. This separation 511 may be provided by an air gap, or in other implementations by an optically transparent liquid, such as water or certain types of oil. Such liquid would typically be retained within a sealed unit (not shown in FIG. 5) at the junction between the first optical light guide 130 and the proximal optical fibre 510. The liquid may be utilised to provide optical matching (based on refractive index) and/or act as a lubricant to support the relative rotation between the proximal end of the first optical light guide 130 and the distal end of the proximal optical fibre 510

As shown in FIG. 5, the drive device 500 includes a motor 540 and first and second gears 520, 530. The first gear 520 is mounted on a drive shaft for rotation by motor 540. The second gear 530, which is mounted coaxially onto the first optical light guide 130, is meshed or otherwise linked (e.g. by frictional coupling) to the first gear 520. When the motor 540 is operated, the first gear 520 rotates, and in turn the second gear 530 is rotated, thereby rotating the first optical fibre 130 and also the ultrasound transmitter 200, 201, 300, 301 located at the distal end of the first optical fibre 130.

In some implementations, the first optical light guide 130 may be housed in a torque coil, also referred to as a torsion coil (not shown in FIG. 5). The torque coil provides accurate transmission of torque along the length of the optical fibre 130, thereby helping to ensure uniform rotation of the optical fibre 130 (without relative twisting along the length of the optical fibre 130). The motor 540 may include an encoder to provide accurate knowledge of the rotational position of the optical fibre 130 (and hence also the ultrasound transmitter) for image reconstruction. Additionally (or alternatively), cross-talk variation between the ultrasound transmitter and ultrasound receiver 155 (such as corresponding to the direct signal mentioned above), can be used to determine the angle of the ultrasound transmitting element relative to the receiver 155. For example, in the configuration (orientation) shown in FIG. 1, the ultrasound beam 171 is directed upwards into the tissue 30, hence the component of beam 171 received as a direct signal at the receiver 155 (rather than as a reflected signal 172) is relatively small (or potentially non-existent). In contrast, if the transmitting element in FIG. 1 were rotated by 180 degrees (about the longitudinal axis) to produce a beam 171 propagating downwards, past the receiver 155, the component of beam 171 received as a direct signal at the receiver 155 is likely to be much higher. Even if the tissue 30 is isotropic, and the receiver is shielded from any direct signal, the received signal obtained by receiver 155 will still generally exhibit some dependency upon the angle of the transmitted beam because the distal end of the instrument lacks rotational symmetry (such as shown in FIG. 4B).

The drive device 500 may be used, for example, in an ultrasound system 10 such as shown in FIG. 1 (in which case the components of FIG. 1 are supplemented by the drive device 500 and the proximal light guide 510). The drive device 500 and proximal light guide may be located at the proximal end of the medical instrument 100, for example, adjacent to (or possibly as part of) coupling device 125. As an example implementation, the drive device 500 may be incorporated into a housing (sled) which can be positioned on or adjacent to the bed of a patient. In addition to providing rotation, in some cases the sled may be moved laterally, such as by a motorised translation stage, to provide three-dimensional imaging (for example, through a helical pull-back). In some cases, the drive device 500 (or housing thereof) may be moved manually to give the operator direct control over positioning of the ultrasound transmitting and receiving elements.

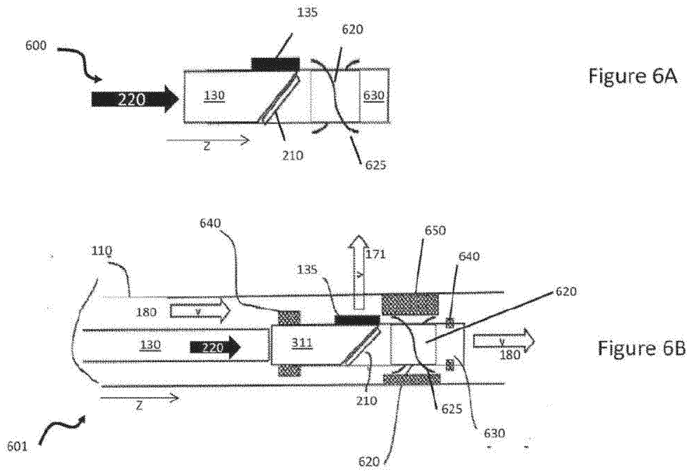

FIG. 6A shows a schematic sectional view of a further example of an ultrasound transmitter 600 for use in a medical instrument 100 such as described in FIG. 1 (note that for clarity, the ultrasound receiver is not shown in this diagram). The transmitter element 600 facilitates the rotation of an ultrasound beam 171 such as discussed above for producing a two dimensional image of the tissue (medium) surrounding the distal end of the probe (for example, a two-dimensional image lying in a plane perpendicular to the axis of rotational scan, which is also generally coincident with the longitudinal axis, z, of the instrument 100).

The ultrasound transmitter 600 includes a first portion which generally corresponds to the ultrasound transmitter 200 shown in FIG. 2A and described above, including an optical light guide 130 with an angled end surface supporting a mirror 210 that reflects excitation light onto an optically absorbing coating 135 to produce an emitted ultrasound beam 171. It will be appreciated that in other implementations, the first portion of transmitter 600 may be based instead on the ultrasound transmitter 201, 300, or 301, such as shown in FIGS. 2B, 3A, or 3B respectively (or any other suitable implementation).

The second portion of the ultrasound transmitter 600, which is located to the distal end of the first portion, is configured to rotate the ultrasound transmitter 600 by using turbine 620 (microturbine) having one or more turbine blades or vanes 625 (shown schematically in FIG. 6A). In particular, a fluid flowing along the longitudinal axis Z of the instrument 100, such as saline solution, provides a force (torque) on the blades 625 to cause them to rotate. The second portion of transmitter 600 further includes an end section 630 which retains the turbine 620 on the instrument 100.

The turbine 620 is positioned after (clear of) the first portion of the ultrasound transmitter 600 so as not to interfere with the transmission of light onto the absorbing surface 135 and the resulting generation of ultrasound. However, in other implementations, the turbine 620 may potentially precede the reflective surface (mirror) 210 and absorbing coating 135, in which case, the body or axial portion at least of the turbine 620 may be substantially transmissive to the excitation light 220.

In the implementation of FIG. 6A, the turbine 620 is typically intended to rotate the whole length of optical fibre 130. However, FIG. 6B shows an alternative implementation in which the turbine 620 only rotates the transmitting element at the distal end of the optical fibre 130, rather than the optical fibre 130 itself. In particular, FIG. 6B shows an example of a transmitter element 601 for use in a medical instrument 100 such as described in FIG. 1. The transmitter element 601 again facilitates the rotation of an ultrasound beam 171 such as discussed above for producing a two dimensional image of the tissue or medium surrounding the distal end of the probe.

The ultrasound transmitter 601 includes a first portion which generally corresponds to the ultrasound transmitter 301 shown in FIG. 3B and described above, including an optical light guide 130 with a perpendicular end surface coupled to an optical head including an additional optical light guide 311 having an angled end surface supporting a mirror 210 that reflects excitation light onto optically absorbing coating 135 to produce an emitted ultrasound beam 171. Accordingly, the first portion of ultrasound transmitter 601 differs slightly from the ultrasound transmitter 301 shown in FIG. 3B, in that rather having a transparent housing 310 that encompasses the distal end of the optical fibre 130 (as shown in FIG. 3B), the ultrasound transmitter 601 has instead an additional optical light guide 311. The additional optical light guide 311 is mechanically separated from the first optical light guide 130 to allow relative rotation between the two. Any physical separation between the two can be filled with air or a transparent liquid as appropriate, for example as discussed in relation to separation 511 of FIG. 5, and may (for example) also provide optical matching and/or lubrication.

The ultrasound transmitter 601 further includes a turbine 620 with blades 625 and an end section 630, which operates substantially as described above in relation to FIG. 6A. In particular, liquid flowing past the turbine within the outer housing 110, such as saline flow 180, rotates the blades 625, and hence also the turbine 620, additional optical light guide 311, and mirror 210, about the longitudinal axis of the instrument, thereby performing an azimuthal scan of the transmitted ultrasound beam (as reflected from mirror 210). FIG. 6B further shows that the ultrasound transmitter 601 includes retaining blocks 640 which act to maintain the position of the additional optical light guide 311 (and hence also turbine 620, which co-rotates with the additional optical light guide 311) with respect to the housing tube 110. The ultrasound transmitter 601 further includes constricting blocks 650 which limit the fluid pathway for the saline 180 to the region of turbine blades 625. In other words, the constricting blocks 650 narrow the passage for the fluid flow, so that the fluid does not pass around (radially outside) the turbine blades 625, but rather engages with these blades 625 for increased efficiency. (It will be appreciated that the retaining blocks 640 and the constricting blocks 650 shown in FIG. 6B are provided by way of example only, and one or both may be omitted in other implementations; likewise, the retaining blocks 640 and the constricting blocks 650 are shown in schematic form only in FIG. 6B and may be implemented, for example, with a more stream-lined shape if so desired).

It will be appreciated that the implementation of FIG. 6B in effect rotationally decouples the optical head including further light guide 311 from the first optical light guide 130. Accordingly, in operation the turbine 620 only has to rotate the further guide light guide 311 at the distal end, rather than all of the first optical light guide 130 (which may extend the full length of the instrument 100).

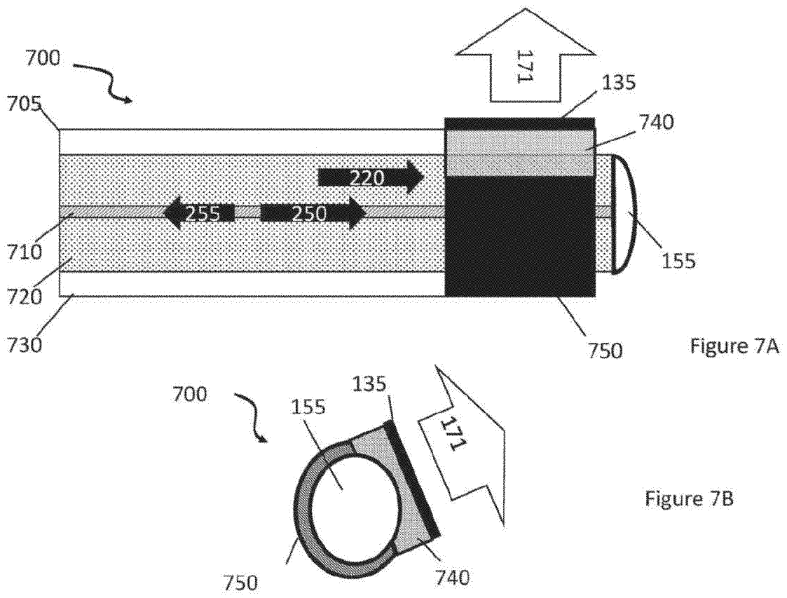

FIG. 7A and FIG. 7B are schematic diagrams showing a side view and an end view, respectively, of an example of a single optical fibre 705 which provides an ultrasound transmitter and receiver pair 700 at the distal end of a medical instrument 100 such as for use in an ultrasound imaging system 10 as described herein. The optical fibre 705 comprises a double clad optical fibre with a single mode core 710. The single mode core 710 carries interrogation light 250 to and reflected interrogation light 255 from optical hydrophone 155. The use of the single mode core for interrogation light 255 reduces the amount of distortion and so preserves an output signal that accurately represents the received ultrasound signal. The optical hydrophone 155 operates substantially as described above in relation to FIG. 4A. Placing the ultrasound receiver (optical hydrophone) 155 at the end of the optical fibre 705 allows omnidirectional reception for all angles of the emitted ultrasound beam 171.

The transmitter-receiver 700 further comprises an inner cladding 720, which guides and retains the single mode light 250/255 in the core 710. The inner cladding 720 further carries (multimode) excitation light 220, which is used for generating ultrasound at the distal end of the instrument 100. Note that this use of multimode for the excitation light increases the amount of excitation light that can be transmitted along the optical fibre 705, and so can help to produce stronger output (and hence stronger reflected) signals, which in turn can lead to images with a higher signal-to-noise ratio.

The transmitter-receiver 700 further comprises an outer cladding 730, which guides and retains the multimode excitation light 220 in the inner cladding 720. The single optical fibre 705 therefore operates as both a transmitting optical fibre (light guide) 130 and as a receiving optical fibre (light guide) 150, such as may be used for the implementation of the ultrasound system 10 of FIG. 1.

As shown in FIG. 7A, the outer cladding 730 is removed from a section of the fibre 705 at the distal end of the instrument 100. One side of this section from which the outer cladding 730 has been removed then receives a mirror (reflective) coating 750. The coating 750 may be formed as a thin metal film, such as gold, which may be coated using evaporation or any other suitable method. The opposing side of the fibre 705 (facing mirror 750) is a coating of an optically transparent material 740 formed, for example, from an epoxy having a high refractive index. This high refractive index causes the excitation light 220 to exit the optical fibre 705 over a relatively short distance. The outside surface of the optical transparent coating 740 is provided with a layer or coating of an optically absorbing material 135. In some examples, the optically absorbing coating 135, may have a shaped profile to control the directionality of the transmitted ultrasound 171.

In operation, the multimode excitation light 220 travelling along through the inner cladding 720 is able to exit fibre 705 where the outer cladding has been removed, and this excitation light passes through transparent coating 740 (in some cases, after reflection by mirror 750) to impinge on (and be absorbed by) optically absorbing material 135. This causes the optically absorbing material 135 to generate an ultrasound beam 171 that propagates into the tissue 30.

The transmitter-receiver device 700 shown in FIGS. 7A and 7B may be integrated into a medical instrument 100 for use with an ultrasound system 10 such as shown in FIG. 1. As described above, the transmitter-receiver device 700 utilises a single dual clad fibre, where the inner cladding is used to guide single mode (interrogation) light to interrogate the ultrasound receiving element 155, and the outer cladding is used to guide multi-mode excitation light for ultrasound generation by optically absorbing coating 135. In some cases, the transmitter-receiver device 700 may be integrated into an over-the-wire catheter which may be placed into a vessel, organ or lumen. When the transmitter-receiver device 700 is rotated, such as by rotating the optical fibre 705 along its length, a two-dimensional ultrasound image (scan) of the surrounding environment may be obtained. This rotation may be achieved, for example, using a configuration similar to that shown in FIG. 5 (in which case the proximal light guide 510 may be implemented as a double clad optical fibre carrying both excitation light 220 and interrogation light 250, in a similar manner to fibre 705).

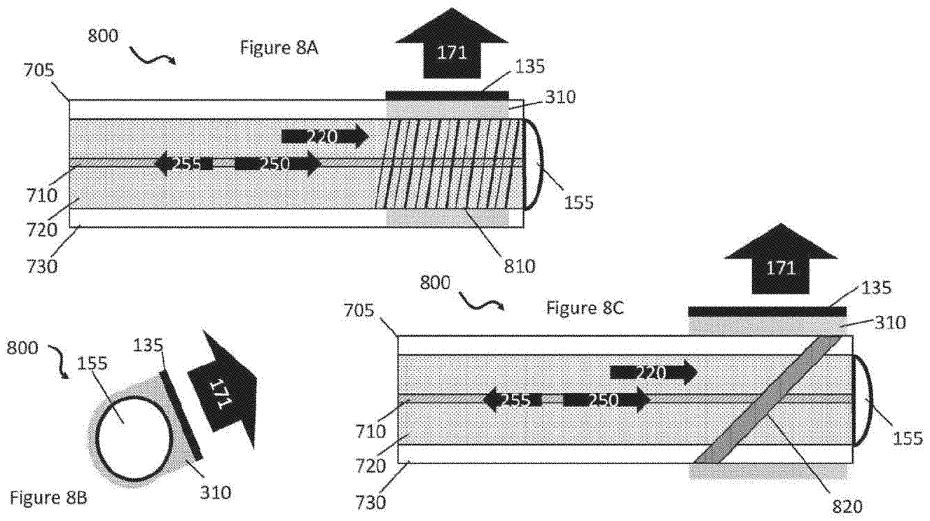

FIG. 8A and FIG. 8B are schematic diagrams showing a side view and an end view, respectively, of another example of a single optical fibre 705 which provides an ultrasound transmitter and receiver pair 800 at the distal end of a medical instrument 100 for use in an ultrasound imaging system 10 as described herein. Many aspects of the example of FIGS. 8A and 8B are the same as for the example shown in FIGS. 7A and 7B, and hence will not be described again (in detail) for reasons of brevity. Note also that these Figures provide (a non-exhaustive) set of examples for redirecting light from (through) the side of a fibre or light guide, and the skilled person will be aware of other suitable methods for redirecting/extracting light from the fibre edge surface.

The integrated transmitter-receiver 800 comprises a double clad optical fibre 705, with a single mode core 710, which carries interrogation light 250 to and from 255 the receiver (optical hydrophone 155), an inner cladding 720, which guides the single mode light 250/255 in the core 710 and carries the multimode excitation light 220 for generation of ultrasound, and an outer cladding 730 which guides the multimode light 220 in the inner cladding 720. The optical hydrophone 155 operates substantially as described above in relation to FIG. 4A. Placing the ultrasound receiver (optical hydrophone) 155 at the end of the optical fibre 705 allows omnidirectional reception for all angles of the emitted ultrasound beam 171. Overall, the single optical fibre 705 therefore operates as both a transmitting optical fibre 130 and as a receiving optical 150.

As shown in FIG. 8A, the outer cladding 730 is removed from a section of the fibre 705 at the distal end of the optical fibre 705 (<0.5 mm from the end) and an angled fibre Bragg grating 810 (shown schematically in FIG. 8A) is formed within this section of the optical fibre 705. (In other implementations, the outer cladding 730 may be retained). The outside of the section of the optical fibre 705 incorporating the angled fibre Bragg grating 810 is coated with an optically transparent housing 310, such as an optical epoxy, and the outside of the optically transparent housing 310 is coated, on one side of the fibre 705, with an optically absorbing coating 135 for producing an ultrasound beam 171. In some examples, the optically absorbing coating 135, may have a shaped profile to control the directionality of the transmitted ultrasound 171.

In operation, the multimode excitation light 220 travels along through the inner cladding 720 to the angled fibre Bragg grating 810. The angled fibre Bragg grating 810 redirects the multimode light 220 through the optically transparent housing 310 towards the optically absorbing coating 135 (this directionality of the angled fibre Bragg grating 810 obviates the need for a mirror such as shown in FIG. 7). The excitation light 220 which impinges on the coating 135 is absorbed and generates an ultrasound beam 171 that propagates into tissue 30. This transmitted ultrasound 171 may be reflected by objects and boundaries in the surrounding environment and hence travel back towards the ultrasound receiver 155.

FIG. 8C shows an alternative implementation of a combined transmitter-receiver 800. As shown in FIG. 8C, an angled dichroic mirror 820 is fabricated between two sections of the fibre 705 which have been cleaved/polished to an angle. Interrogation light travelling to 250 and from 255 the receiver 155, is transmitted by the angled mirror 810, whilst multimode excitation light 220 is reflected by the mirror 820. The outside of the section of the optical fibre 705 incorporating the angled dichroic mirror 820 is coated with an optically transparent housing 310, such as an optical epoxy, and the outside of the optically transparent housing 310 is coated, on one side of the fibre 705, with an optically absorbing coating 135 for producing an ultrasound beam 171. In some examples, the optically absorbing coating 135, may have a shaped profile to control the directionality of the transmitted ultrasound 171.

In operation, the multimode excitation light 220 travels along through the inner cladding 720 to the angled dichroic mirror 820. The angled dichroic mirror 820 redirects the multimode light 220 through the optically transparent housing 310 towards the optically absorbing coating 135 (this directionality of the angled dichroic mirror 820 obviates the need for a further mirror coating such as shown in FIG. 7). The excitation light 220 which impinges on the coating 135 is absorbed and generates an ultrasound beam 171 that propagates into tissue 30. This transmitted ultrasound 171 may be reflected by objects and boundaries in the surrounding environment and hence travel back towards the ultrasound receiver 155.

The combined transmitter-receiver 800 may be integrated into a medical instrument 100 for use with an ultrasound system 10 such as shown in FIG. 1. As described above, the transmitter-receiver 800 utilises a single dual clad fibre, where the inner cladding is used to guide single mode (interrogation) light to interrogate the ultrasound receiving element 155, and the outer cladding is used to guide multi-mode excitation light for ultrasound generation by an optically absorbing coating 135. In some cases, the transmitter-receiver 800 may be integrated into an over-the-wire catheter which may be placed into a vessel, organ or lumen, or formed as part of a very thin guidewire. When the transmitter-receiver 800 is rotated, such as by rotating the optical fibre 705 along its length, a two-dimensional ultrasound image (scan) of the surrounding environment may be obtained. This rotation may again be achieved, for example, using a configuration similar to that shown in FIG. 5 (in which case the proximal light guide 510 may be implemented as a double clad optical fibre carrying both excitation light 220 and interrogation light 250, in a similar manner to fibre 705).

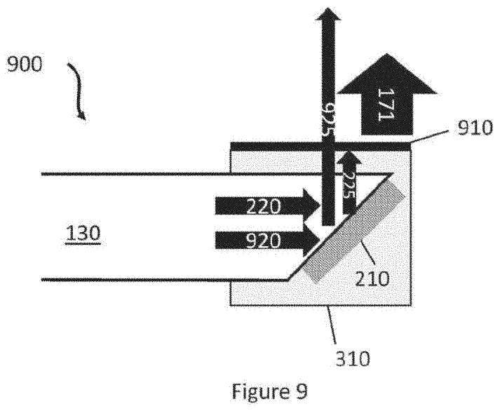

FIG. 9 is a schematic diagram of an example of a dual-modality ultrasound and photoacoustic transmitter 900 for use in a medical instrument in accordance with some implementations of the approach described herein (note that for clarity, the ultrasound receiver is not shown in this diagram). The transmitter 900 comprises an optical fibre 130 that serves as the excitation light guide. The distal end of the optical fibre 130 is provided with an angled (45°) surface which is coated with a mirror 210. The mirror 210 may be implemented, for example, using a metallic film such as gold, and may be formed, for example, by using evaporation, sputtering or any other suitable technique.

A housing 310 is formed round the distal end of the fibre 130. This housing may be made from an optically transparent epoxy, such as NOR81 (Norland). The housing 310 is itself provided (at least in the part opposing mirror 210) with a coating 910 of a wavelength selective optically absorbing medium. For example, this coating 910 may be a composite of polydimethylsiloxane, used for its high thermal expansion coefficient, and a selectively absorbing material, such as gold nanoparticles. The coating 910 may be applied to the housing 310 by dip coating or by any other suitable method.

In operation, the excitation light 220 travels along the optical fibre 130 to the distal end of the transmitter 900. The mirror 210 is configured to reflect this excitation light 220 to form reflected excitation light 225, which exits the side of the fibre 130 to travel into and through the housing 310. The redirected (reflected) light 225 then impinges upon the selectively optically absorbing coating 910, which is configured to absorb the reflected light in order to generate ultrasound waves 171 that propagate away from the transmitter 900 into the tissue.

FIG. 9 also shows light 920 having a different wavelength from the excitation light 220 travelling along the optical fibre 130 and impinging upon the mirror 210. This light 920 is reflected by mirror 210 to create reflected light 925, which propagates not only through the optically transparent housing, but also through the wavelength selective coating 910 and into the surrounding tissue 30. In other words, the absorbing coating 910 is configured to have a wavelength-dependent absorption profile such that wavelengths corresponding to the excitation light 220 are absorbed by the coating 910m while wavelengths corresponding to the other light 920 are transmitted through the absorbing surface 910 and into tissue as an optical (rather than ultrasound) input.

In some implementations, the optically absorbing coating 910, may have a shaped profile to focus or otherwise direct the transmitted ultrasound 171. In some examples, the epoxy housing 310 may have a shaped profile, or graded refractive index, which can be used to help focus or direct the transmitted light 925.

The wavelength-selective absorbing coating 910 on the transmitter 900 allows both ultrasound and optical energy to be transmitted into the tissue 30. The ultrasound waves 171 transmitted into tissue 30 may be used for ultrasound imaging as described above. The optical waves transmitted into the tissue may be used for various purposes. For example, in some cases, the optical energy may be used for laser ablation, while in other cases the light 925 is absorbed within the surrounding medium to generate ultrasound via the photoacoustic effect. This ultrasound generated within the tissue may be detected by the receiver provided in the medical instrument (not shown in FIG. 9) for imaging and/or monitoring purposes (for example).

Note that excitation light 220 may have a first wavelength and excitation light 920 has a second wavelength different from the first wavelength. In this context, the first wavelength may correspond to a single wavelength, for a monochromatic source, or may correspond to a range or band of wavelengths, likewise for the second wavelength. The coating 910 (or other coatings described herein) may be regarded as optically absorbing if they absorb more than 50%, more than 75%, more than 90%, or more than 95% of the incident light of a given wavelength (whether corresponding to a monochromatic source or a wavelength band); likewise the coating 910 (or other coatings described herein) may be regarded as optically transmissive if they transmit more than 50%, more than 75%, more than 90%, or more than 95% of the incident light of a given wavelength.

The transmitter 900 may be integrated into a medical instrument 100 for use with an ultrasound system 10 such as shown in FIG. 1. In some cases, the transmitter 900 may be integrated into an over-the-wire catheter which may be placed into a vessel, organ or lumen, or formed as part of a very thin guidewire. When the transmitter 900 is rotated, such as by rotating the light guide 130 along its length, a two-dimensional ultrasound image (scan) of the surrounding environment may be obtained. This rotation may be achieved, for example, using a configuration similar to that shown in FIG. 5. Note that in some implementations, the provision of light 920 may be synchronised with the rotation of light guide 130, such that the light 920 is only transmitted within a limited range of azimuthal angles with respect to the main longitudinal axis of the instrument 100; this could be used, for example, to provide a laser ablation beam that is targeted to a particular region of tissue.

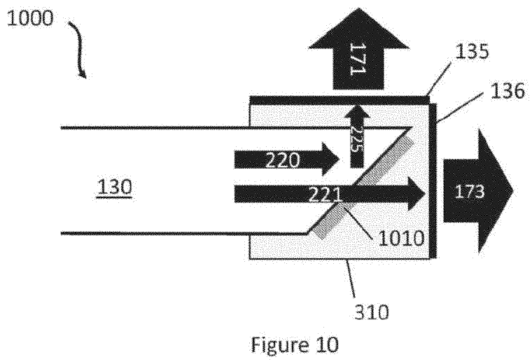

FIG. 10 is a schematic diagram of another example of an ultrasound transmitter 1000 for use in a medical instrument in accordance with some implementations of the approach described herein (note that for clarity, the ultrasound receiver is not shown in this diagram). The transmitter 1000 comprises an optical fibre 130 that serves as the excitation light guide. The distal end of the optical fibre 130 is provided with an angled (45°) surface which is coated with a dichroic mirror 1010. The mirror 1010 may be formed, for example, by a dielectric coating.

A housing 310 is formed round the distal end of the fibre 130. This housing may be made from an optically transparent epoxy, such as NOR81 (Norland). The housing 310 is itself provided (at least in the part facing mirror 1010) with a coating 135 of an optically absorbing medium. For example, this coating 135 may be a composite of polydimethylsiloxane, used for its high thermal expansion coefficient, and an absorbing material, such carbon nanotubes. The coating 135 may be applied to the housing 310 by dip coating or by any other suitable method. A second optically absorbing coating 136 is applied to the distal end of the housing 310.

In operation, the excitation light 220 travels along the optical fibre 130 to the distal end of the transmitter 1000. The mirror 1010 is configured to reflect light in the wavelength range of the excitation light 220, thereby forming reflected excitation light 225. The redirected light 225 leaves through the edge of the optical fibre 130 and impinges upon the optically absorbing coating 135, where it is absorbed and leads to the generation of an ultrasound wave 171, which propagates away from the transmitter 1000 into the tissue 30.

FIG. 10 also shows light 221 having a different wavelength from the excitation light 220 travelling along the optical fibre 130 and impinging upon the coating 1010, which is transparent to light of this different wavelength. The light 221 is therefore transmitted by the dichroic mirror 1010 towards coating 136, and is then absorbed within the coating 136 to generate ultrasound 173. This ultrasound beam propagates away from the transmitter 1000 in a forward direction, i.e. along the longitudinal axis of the device (in contrast to the radial direction of ultrasound beam 171). In some examples, either or both of the optically absorbing coatings 135, 136, may have a shaped profile to focus or otherwise control the direction of the transmitted ultrasound 171, 173.

The transmitter 1000 may be integrated into a medical instrument 100 for use with an ultrasound system 10 such as shown in FIG. 1. In some cases, the transmitter 1000 may be integrated into an over-the-wire catheter which may be placed into a vessel, organ or lumen, or formed as part of a very thin guidewire. When the transmitter 1000 is rotated, such as by rotating the light guide 130 along its length, a two-dimensional ultrasound image (scan) of the surrounding environment may be obtained, as described above. This rotation may be achieved, for example, using a configuration similar to that shown in FIG. 5. The ultrasound beam 173 may be used to give a look-ahead (axial) view, in addition to the sideways (radial) scan view from ultrasound beam 171. This further information may be useful, for example, for guiding the insertion of the medical instrument 100 into tissue. The reflected signal from ultrasound beam 173 may be received by the same receiver as used for ultrasound beam 171, such as an optical hydrophone 155. The received signals may be distinguished by suitable multiplexing of the ultrasound beams 171, 173, for example in terms of frequency (whereby ultrasound beam 171 has a different frequency from ultrasound beam 173) and/or temporally (whereby ultrasound beam 171 is emitted at different times from ultrasound beam 173, or with a different temporal pattern).

In some implementations, one or both of coatings 135, 136 may be dichroic (wavelength selective), analogous to the implementation of FIG. 9, so as to additionally support the provision of at least one optical beam into the tissue 30. Such an optical beam would then allow transmitter 1000 to offer further functionality, such as laser ablation and dual-modality image acquisition based firstly on ultrasound and secondly on the photoacoustic effect.

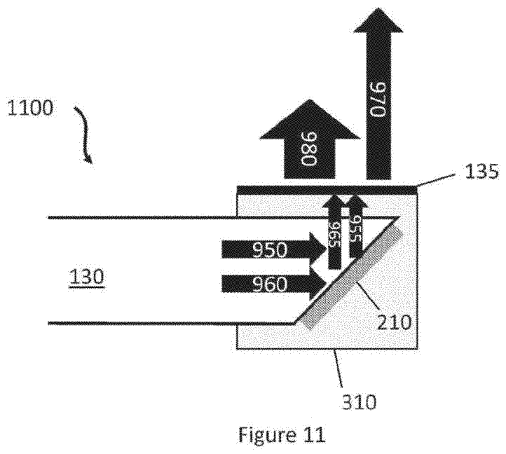

FIG. 11 is a schematic diagram of another example of an ultrasound transmitter 1100 for use in a medical instrument in accordance with some implementations of the approach described herein (note that for clarity, the ultrasound receiver is not shown in this diagram). The transmitter 1100 comprises an optical fibre 130 that serves as the excitation light guide. The distal end of the optical fibre 130 is provided with an angled (45°) surface which is coated with a dichroic mirror 210. The mirror 210 may be implemented, for example, using a metallic film such as gold, and may be formed, for example, by using evaporation, sputtering or any other suitable technique.

A housing 310 is formed round the distal end of the fibre 130. This housing may be made from an optically transparent epoxy, such as NOR81 (Norland). The housing 310 is itself provided (at least in the part facing mirror 210) with a coating 135 of an optically absorbing medium (material). For example, this coating 135 may be a composite of polydimethylsiloxane, used for its high thermal expansion coefficient, and an absorbing material, such carbon nanotubes. The coating 135 may be applied to the housing 310 by dip coating or by any other suitable method.

Note that the general configuration shown in FIG. 11 matches the configuration shown in FIG. 3A (and is also similar to the configuration shown in FIG. 9, with the exception that in FIG. 9 the optically absorbing coating 910 is dichroic, whereas the optically absorbing coating 135 shown in FIGS. 3A and 11 does not have to be dichroic). However, FIG. 11 shows an additional way of using transmitter 1100, in particular demonstrating the use of multiple optical excitation regimes to provide different ultrasonic outputs.

In operation, two beams of excitation light 950, 960 travel along the optical fibre 130 to the distal end of the transmitter 1100. The mirror 210 is configured to reflect excitation light 950 to form reflected excitation light 955, which exits the side of the fibre 130 to travel into and through the housing 310, and also to reflect excitation light 960 to form reflected excitation light 965, which likewise exits the side of the fibre 130 to travel into and through the housing 310. The two beams of redirected (reflected) light 955, 965 then impinge upon the optically absorbing coating 135, which is configured to absorb the reflected light and hence to generate ultrasound beam 970 (from reflected light 955) and also ultrasound beam 980 (from reflected light 965), such that both ultrasound beams 970, 980 then propagate away from the transmitter 900 into the tissue.

One reason for generating two different ultrasound beams 970, 980 in this manner is for the two ultrasound beams 970, 980 to have different wavelengths or frequencies. Thus lower frequency ultrasound waves tend to propagate further in tissue than higher frequency ultrasound waves. Accordingly, the latter are better suited to imaging closer to the transmitter (in relatively high resolution, because of the shorter ultrasound wavelength), while the former are better suited to imaging further away from the transmitter 1100. Combining the results obtained at the different frequencies therefore allows a more complete (extensive) composite ultrasound image to be obtained.

In order to support such imaging, the two beams of excitation light 950, 960 may have different (first and second) modulation patterns applied to them. These first and second modulation patterns are then carried over into the ultrasound beams 970, 980 respectively generated from these two beams of excitation light 950, 960, and likewise into the received (reflected) ultrasound signals produced by the ultrasound beams 970, 980.

For example, in one implementation, excitation light 950 may have a relatively low modulation frequency, such as provided by a chirped or long pulse having a bandwidth of 1 to 5 MHz. This type of modulation can be readily implemented using (for example) a diode laser. As noted above, the corresponding ultrasound beam 970 is governed by the temporal profile of the absorbed light 955, and so likewise has a relatively low (ultrasound) frequency (1 to 5 MHz), which has a relatively low absorption coefficient in most media, and so can generally penetrate further to give higher depth imaging.

In contrast, excitation light 960 may have a relatively high modulation frequency, such as provided by a chirped or short pulse having a bandwidth of 20 to 40 MHz. This type of modulation can again be readily implemented using (for example) a diode laser. The corresponding ultrasound beam 980 is governed by the temporal profile of the absorbed light 965, and so likewise has a relatively high (ultrasound) frequency (20 to 40 MHz), which has a higher absorption coefficient in most media. Accordingly, such high frequency ultrasound generally cannot penetrate as far into tissue as the lower frequency ultrasound, but the higher frequency ultrasound provides higher resolution (because of the shorter wavelength). Consequently, the ultrasound transmitter 1100 can be used to acquire imaging of two or more depth ranges (depending on the number of different excitation beams provided).

The transmitter 1100 may be integrated into a medical instrument 100 for use with an ultrasound system 10 such as shown in FIG. 1. In some cases, the transmitter 1100 may be integrated into an over-the-wire catheter which may be placed into a vessel, organ or lumen, or formed as part of a very thin guidewire. When the transmitter 1100 is rotated, such as by rotating the light guide 130 along its length, a two-dimensional ultrasound image (scan) of the surrounding environment may be obtained, as described above, based on ultrasound beams 970 and 980. This rotation may be achieved, for example, using a configuration similar to that shown in FIG. 5.

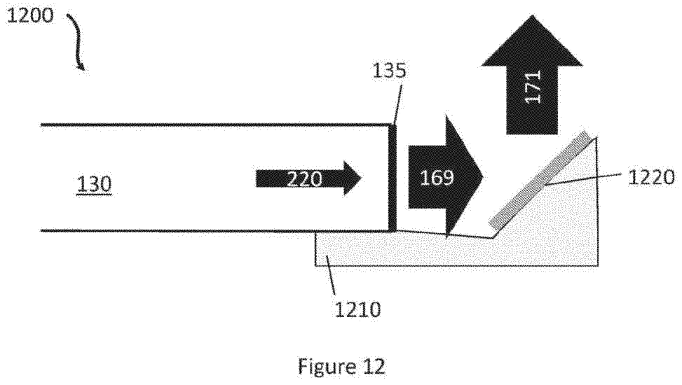

FIG. 12 is a schematic diagram of another example of an ultrasound transmitter 1200 for use in a medical instrument in accordance with some implementations of the approach described herein (note that for clarity, the ultrasound receiver is not shown in this diagram). The transmitter 1200 comprises an optical fibre 130 that serves as the excitation light guide. The distal end of the optical fibre 130 is provided with a coating 135 of an optically absorbing medium. For example, this coating 135 may be a composite of polydimethylsiloxane, used for its high thermal expansion coefficient, and an absorbing material, such carbon nanotubes. The coating 135 may be applied to the housing 310 by dip coating or by any other suitable method.

Attached to the distal end 103 of the optical fibre 130 is a mount 1210, which may be made from epoxy or other suitable materials. The mount 1210 supports an acoustic mirror (acoustic deflector or reflector) 1220 positioned at an angle of 45° relative to the longitudinal axis of the optical fibre 130. In operation, excitation light 220 travels along the optical fibre 130 to the distal end of the transmitter 1200, where the excitation light enters and is absorbed by the coating 135 to generate an ultrasound beam 169 in a substantially axial direction, i.e. parallel to the main longitudinal axis of the device. The ultrasound beam 169 then propagates towards the acoustic mirror 1220, which reflects the ultrasound beam 169 in a substantially radial direction, i.e. perpendicular to the main longitudinal axis of the device. The reflected ultrasound beam, denoted by reference numeral 171 then propagates into tissue for performing ultrasound imaging in a generally similar manner to that described above, e.g. for the implementations shown in FIGS. 3A and 3B.

In some implementations, the optically absorbing coating 135 may be shaped or profiled to focus or otherwise control the direction of the transmitted ultrasound 169. Alternatively (or additionally), the acoustic mirror 1220 can be shaped or profiled to provide acoustic focusing (or other shaping) of the reflected ultrasound beam 171. In addition, in some implementations, the acoustic mirror 1220 may be set at an offset angle other than 45°, thereby reflecting the ultrasound beam 171 at a different angle away from the longitudinal axis of the transmitter 1200 (in a direction intermediate radial and axial).

In some implementations, the mount 1210 and the acoustic mirror 1220 may be physically detached from the optical fibre 130 and the absorbing coating 135. For example, they may be supported within an outer housing (not shown in FIG. 12), analogous to the configuration shown in FIG. 6B. Such a configuration allows the mount 1210 and mirror 1220 to be rotated independently of the optical fibre 130, thereby angularly rotating the direction of the reflected ultrasound 171 about the longitudinal axis of the transmitter 1200.

The transmitter device 1200 shown in FIG. 12 may be integrated into a medical instrument 100 for use with an ultrasound system 10 such as shown in FIG. 1. For example, the device may be used with a thin guide wire or integrated into an over-the-wire catheter which may be placed into a vessel, organ or lumen, paired with a receiver for performing ultrasound imaging. When the transmitter 1200 is rotated, such as by rotating the optical fibre 130 along its length (or potentially just rotating mount 1210 and mirror 1220), a two-dimensional ultrasound image (scan) of the surrounding environment may be obtained. This rotation of the optical fibre 130 may be achieved, for example, using a configuration similar to that shown in FIG. 5.

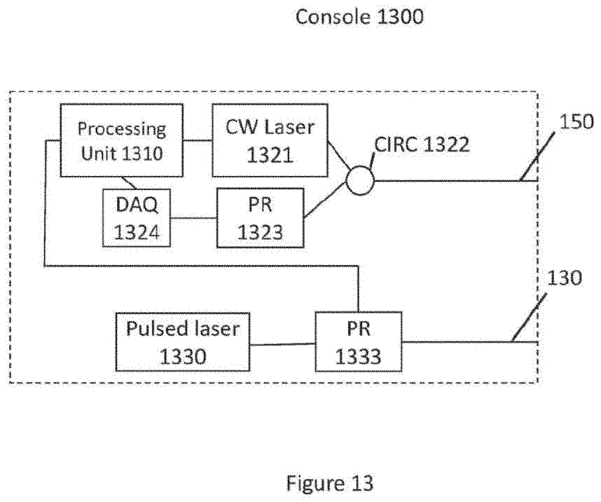

FIG. 13 is a schematic diagram showing a console 1300 such as for use in the ultrasound system 10 of FIG. 1. The console 1300 can be considered as providing the components at the proximal end 102 of the medical instrument (ultrasound probe) 100 (for reasons of clarity, not all of these components are shown in FIG. 1). The console 1300 components may for example be provided on a trolley or bedside table for interconnection to the ultrasound probe during a clinical procedure. The console 1300 may include a display (not shown), such as display 21 from FIG. 1. The processing unit 1310 of the console may be implemented as part of the processing unit 20 shown in FIG. 1 (or ultrasound system 10 may be provided with multiple processing units).

In the implementation shown in FIG. 13, the console 1300 includes a pulsed laser source 1330 which is linked to the first optical light guide 130 (the ultrasound transmitter optical fibre) 130 via a photoreceiver 1333. The pulsed laser source 1330 is used to provide the excitation light 220 (the excitation light could also be provided as appropriate by various other light sources, such as a modulated diode laser, for example). The console 1300 further includes a continuous wave tuneable laser 1321 which is used to provide the interrogation light 250. The continuous wave tuneable laser 1321 is connected to the second optical light guide 150 (the ultrasound receiver optical fibre) via a circulator 1322. The second optical light guide 150 is also connected to a photoreceiver 1323 via the circulator 1322. The output of the photoreceiver 1323 is connected one or more data acquisition cards 1324. The output from the photoreceiver 1333 is used to synchronise the receiver system to the ultrasound generation (excitation light) laser 1330 output.

In operation of an ultrasound system 10 including the console 1300, interrogation light 250 is delivered into the second optical light guide (ultrasound receiver fibre) 150 via the circulator 1322, while reflected light 255 from the receiver 155 (not shown in FIG. 13) is delivered to the photoreceiver 1323 via the circulator 1322. In some implementations, the photoreceiver 1323 may split the received signal into low (e.g. <50 kHz) and high (>500 kHz) frequency components, which may be digitised using separate data acquisition cards 1324. The low frequency signal component is then used to bias a Fabry-Pérot sensor provided in the receiver 155, while the high frequency signal component is encoded with the received ultrasound signal from receiver 155.

As described herein, an ultrasound probe comprises an optical light guide comprising a multi-mode optical waveguide for transmitting excitation light and a single-mode optical waveguide for transmitting interrogation light. The multi-mode waveguide and the single-mode waveguide may be provided, for example, in the same or in separate optical fibres. In the latter case, the optical light guide may comprise two (or more) optical fibres, where at least one of the optical fibres has a single mode core and another optical fibre has a multimode core.

The probe further comprises an ultrasound transmitter located at a distal end of the probe, the ultrasound transmitter comprising an optically absorbing material for absorbing the excitation light from the multi-mode optical waveguide to generate an ultrasound beam via the photoacoustic effect. The probe further comprises an ultrasound receiver including an optical cavity external to the single-mode optical waveguide. The interrogation light from the single-mode optical waveguide is provided to the ultrasound receiver. The optical cavity has a reflectivity that is modulated by impinging ultrasound waves. The interrogation light is reflected from the optical cavity to a proximal end of the single-mode optical waveguide where it can be received as a signal representative of the ultrasound incident on the receiver.

At least a portion of the ultrasound probe is configured to rotate so that the ultrasound beam is transmitted in a rotating direction. The direction of the ultrasound beam may be determined according to the centre or centroid of the beam, or by any other suitable measure. This direction is rotated about an axis, and the ultrasound beam moves (rotates) to stay alignment with the direction (as it rotates). For example, the ultrasound probe may be configured to transmit the ultrasound beam away from a longitudinal axis of the probe in a direction which is rotated about the longitudinal axis.

In some cases, the direction of the ultrasound beam is perpendicular to the axis of rotation. Accordingly, the rotating beam can be considered as defining (sweeping out) a flat planar surface having a circular configuration (analogous to a lighthouse). This perpendicular arrangement usually provides the shortest travel distance from the probe to an object being imaged (for all distances from the probe), which can help to improve signal strength.

In other implementations, the beam direction may have an angle which is offset from (less than) ninety degrees to the axis, so that the beam sweeps out the surface of a cone. There are various possible reasons for such a configuration. For example, the ultrasound transmitter and receiver may be longitudinally separated from one another, so this offset angle can be selected to accommodate this longitudinal separation. Another possibility is that an object of interest may be somewhat hidden behind another object, and the offset angle may allow a stronger ultrasound signal to reach the object of interest (depending upon the details of the geometry).

There are a variety of ways in which the rotating ultrasound beam can be produced. For example, the ultrasound probe is configured to rotate the optically absorbing material about the longitudinal axis of the probe, and/or the ultrasound probe is configured to rotate an optical reflector to transmit the excitation light away from the longitudinal axis of the probe of the probe in a direction which is rotated about the longitudinal axis (such as shown in FIG. 2A). In some cases, the rotation of the optically absorbing material is synchronised with the rotation of the optical reflector, so that the reflected excitation light continues to impact the optically absorbing material; note that this may involve rotation of the entire probe. In another configuration, the optically absorbing material may be arranged in an azimuthal band (such as shown in FIG. 2B). This band does not need to rotate, rather the excitation light beam rotates around the band of optically absorbing material to generate the rotating ultrasound beam. In another configuration, an azimuth band of excitation light is produced, which therefore does not need to rotate, and this is then used in conjunction with a rotating optically absorbing medium (which is illuminated by the excitation light at any angle) to produce the rotating ultrasound beam. In another configuration, such as shown in FIG. 12, the ultrasound beam itself is produced in an initially longitudinal direction along the longitudinal axis of the probe, and a rotating acoustic reflector is provided to deflect the ultrasound beam away from the longitudinal axis of the probe. Other configurations will be apparent to the skilled person.

In some implementations, such as shown in FIG. 6B, the ultrasound probe may further comprise an optical head located at the distal end of the probe. The optical head may include the ultrasound transmitter and/or the ultrasound receiver. The optical head may be configured to rotate relative to the optical light guide, for example by providing a micro-turbine and photo-receptors configured to rotate the optical head in response to incident light. In such a configuration, there is no need to rotate the whole length of the optical light guide in order to rotate components in the optical head.

In some implementations, such as shown in FIGS. 7A and 8A, the optical light guide comprises a double clad optical fibre having an inner cladding to form the multimode waveguide (core) for the excitation light. The double clad optical fibre may include an inner cladding layer, whereby at least a portion of the excitation light may be extracted via the inner cladding layer at the distal end of the probe. In such a configuration, an optical element may be provided to redirect excitation light from the inner cladding to the optically absorbing material. Additionally, or alternatively, a portion of high refractive index optical epoxy may be located between the multimode core and the optically absorbing material to draw excitation light out of the optical fibre (light guide). In some implementations, such as shown in FIG. 10A, a Bragg grating located in the multimode core is used to redirect excitation light out of the optical fibre towards the optically absorbing material.

In some implementations, the optically absorbing material may be substantially opaque to the excitation light having a first wavelength, and substantially transparent to light having a second wavelength which is emitted from the ultrasound probe (such as shown in FIG. 9). In such a configuration, the light having the second wavelength may be used (for example) for producing the photoacoustic effect in the tissue around the probe. In a variation on this approach, such as shown in FIG. 10, an optical element may be used for redirecting excitation light having a first wavelength to a first region of the optically absorbing material, and for transmitting light having a second wavelength to a second region of optically absorbing material. The first region may be angled with respect to the second region. For example, in FIG. 10, the first region is used to produce an ultrasound beam which is perpendicular to the longitudinal axis of the probe, while the second region is used to produce a longitudinally-aligned beam.

The ultrasound probe described herein may be incorporated into a medical instrument and/or an ultrasound system. The latter may further include a console for receiving the signal from the ultrasound probe as a function of angle of rotation of the ultrasound beam.

In conclusion, various implementations have been described herein, by way of example only, and without limitation. It will be appreciated by the skilled person that features of different implementations can generally be combined with one another to create new implementations. Accordingly, the scope of the application is not restricted to particular examples or implementations described herein, but rather is defined by the appended claims and equivalents.

1. An ultrasound probe comprising:
an optical light guide comprising a multi-mode optical waveguide for transmitting excitation light and a single-mode optical waveguide for transmitting interrogation light;
an ultrasound transmitter located at a distal end of the probe, the ultrasound transmitter comprising an optically absorbing material for absorbing the excitation light from the multi-mode optical waveguide to generate an ultrasound beam via the photoacoustic effect; and
an ultrasound receiver including an optical cavity external to the single-mode optical waveguide to which the interrogation light from the single-mode optical waveguide is provided, the optical cavity having a reflectivity that is modulated by impinging ultrasound waves, wherein interrogation light reflected from the optical cavity to a proximal end of the single-mode optical waveguide is received for generating a signal; and
wherein at least a portion of the ultrasound probe is configured to rotate so that the ultrasound beam is transmitted in a rotating direction.

2. The ultrasound probe of claim 1, wherein the ultrasound probe is configured to transmit the ultrasound beam away from a longitudinal axis of the probe in a direction which is rotated about the longitudinal axis.

3. The ultrasound probe of claim 2, wherein the ultrasound probe is configured to rotate the optically absorbing material about the longitudinal axis of the probe,
wherein the ultrasound probe is configured to rotate an optical reflector to transmit the excitation light away from the longitudinal axis of the probe of the probe in a direction which is rotated about the longitudinal axis,
wherein the rotation of the optically absorbing material is synchronised with the rotation of the optical reflector.

4. The ultrasound probe of claim 2, wherein the ultrasound probe is configured to rotate an optical reflector to transmit the excitation light away from the longitudinal axis of the probe of the probe in a direction which is rotated about the longitudinal axis.

5. (canceled)

6. The ultrasound probe of claim 2, wherein the ultrasound probe is configured to rotate an acoustic reflector to deflect the ultrasound beam away from the longitudinal axis of the probe.

7. The ultrasound probe of claim 2, wherein the direction of the ultrasound beam is perpendicular to the longitudinal axis of the probe,
wherein the ultrasound receiver is substantially isotropic in sensitivity in a frequency range used for imaging.

8. The ultrasound probe of claim 1, further comprising a torsion coil for rotating the optical light guide.

9. The ultrasound probe of claim 1, further comprising an optical head located at the distal end of the probe, the optical head including the ultrasound transmitter and being configured to rotate relative to the optical light guide.

10. The ultrasound probe of claim 9, wherein the optical head comprises a micro-turbine and photo-receptors configured to rotate the optical head in response to incident light.

11. (canceled)

12. The ultrasound probe of claim 1, wherein the ultrasound probe is further configured to determine a rotation angle for the direction of the ultrasound beam using ultrasound cross-talk between the ultrasound transmitter and the ultrasound receiver.

13. The ultrasound probe of claim 12, wherein the ultrasound cross-talk includes a direct component propagating from the ultrasound transmitter to the ultrasound receiver without reflection in a medium surrounding the ultrasound probe.

14. The ultrasound probe of claim 1, wherein the ultrasound receiver comprises a Fabry-Pérot cavity,
wherein the optical light guide comprises a double clad optical fibre having an inner cladding to form the multi-mode waveguide for the excitation light,
wherein the double clad optical fibre includes an inner cladding layer, and wherein at least a portion of the excitation light is extracted via the inner cladding layer at the distal end of the probe,
the ultrasound probe further comprising an optical element to redirect excitation light from the inner cladding to the optically absorbing material,
wherein the optical element is a fibre Bragg grating.

15. (canceled)

16. (canceled)

17. (canceled)

18. (canceled)

19. The ultrasound probe of claim 1, wherein the optical light guide comprises a double clad optical fibre having an inner cladding to form the multi-mode waveguide for the excitation light,
the ultrasound probe further comprising an optical element to redirect excitation light from the inner cladding to the optically absorbing material,
wherein the optical element is an angled dichroic mirror between a proximal section of double-clad fibre and a distal section of either double-clad or single-mode optical fibre.

20. The ultrasound probe of claim 1, wherein the optically absorbing material is substantially opaque to the excitation light having a first wavelength, and substantially transparent to light having a second wavelength which is emitted from the ultrasound probe.

21. The ultrasound probe of claim 1, further comprising an optical element for redirecting excitation light having a first wavelength to a first region of the optically absorbing material, and for transmitting light having a second wavelength to a second region of optically absorbing material, the first region being angled with respect to the second region.

22. A medical instrument incorporating the ultrasound probe of claim 1.

23. An ultrasound system including the ultrasound probe of claim 1 and a console, wherein said console is configured received said signal from the ultrasound probe as a function of angle of rotation of the direction of the transmitted ultrasound beam,
wherein the console is configured to translate the ultrasound transmitter and/or the ultrasound receiver along a longitudinal axis of the ultrasound probe, and to combine signals received at different respective translations to form an image.

24. (canceled)

25. An ultrasound system including the ultrasound probe of claim 1 and a console, wherein said console is configured to receive said signal from the ultrasound probe as a function of the angle of rotation of the transmitted ultrasound beam, wherein the console is configure to apply different filters to the received signals to generate a plurality of filtered signals, and to combine the filtered signals to form an image.

26. The ultrasound system of claim 23, wherein the console is configured to provide excitation light pulses of different durations to generate ultrasound at different frequency ranges,
wherein received signals acquired with different excitation light pulse durations are combined to generate an image,
wherein the excitation light pulses comprise waveforms with low autocorrelations,
wherein the durations of the excitation light pulses are determined based on previously received signals.

27. (canceled)

28. (canceled)

29. (canceled)

30. An ultrasound system including the ultrasound probe of claim 1 and a console, wherein said console is configured to receive said signal from the ultrasound probe as a function of the angle of rotation of the transmitted ultrasound beam,
wherein the console is coupled to the optical light guide by an optic rotary junction.
